(19) United States
(12) Patent Application Publication
(10) Pub. No.: US 2026/0144762 A1
(43) Pub. Date: May 28, 2026

(54) TRANSDERMAL DELIVERY SYSTEM WITH A MICROPOROUS MEMBRANE HAVING SOLVENT-FILLED PORES

(71) Applicant: Corium, LLC, Cambridge, MA (US)

(72) Inventors: Eun Soo Lee, Redwood City, CA (US); Amit K. Jain, Milpitas, CA (US); Parminder Singh, Union City, CA (US)

(21) Appl. No.: 19/180,842

(22) Filed: Apr. 16, 2025

Related U.S. Application Data
(63) Continuation of application No. 18/298,623, filed on Apr. 11, 2023, now abandoned.
Application No. 18/298,623 is a continuation of application No. 16/046,952, filed on Jul. 26, 2018, now abandoned.
(60) Provisional application No. 62/537,414, filed on Jul. 26, 2017.

(51) Int. Cl. A61K 9/70; A61K 31/13; A61K 31/445; A61K 47/02; A61K 47/10; A61K 47/12; A61K 47/26; A61K 47/32; A61P 25/28

(52) U.S. Cl. CPC A61K 9/7084; A61K 9/7061; A61K 31/13; A61K 31/445; A61K 47/02; A61K 47/10; A61K 47/12; A61K 47/26; A61K 47/32; A61P 25/28

(57) ABSTRACT

A transdermal delivery system is described, where the system comprises a drug reservoir layer comprising an active agent and a skin contact adhesive layer. A microporous membrane that has been pretreated with a membrane treatment composition before the membrane is incorporated into the system is disposed between the drug reservoir layer and the skin contact adhesive layer.

### CROSS-REFERENCE TO RELATED APPLICATIONS

This application is a continuation of U.S. application Ser. No. 18/298,623, filed Apr. 11, 2023, which is a continuation of U.S. application Ser. No. 16/046,952, filed Jul. 26, 2018, which claims priority to U.S. Provisional Application No. 62/537,414, filed Jul. 26, 2017, each of which is incorporated herein in its entirety for all purposes.

### TECHNICAL FIELD

The subject matter described herein relates to transdermal delivery systems for delivery of an active agent in which the systems include a microporous membrane having pores that include a membrane treatment composition.

### BACKGROUND

Transdermal drug delivery systems can be an effective means for administering active pharmaceutical agents that might have disadvantages when administered via other routes such as orally or parenterally. However, the delivery of many drugs over a long period of time (e.g. several days or more) is difficult. Transdermal delivery of basic (i.e., alkaline) drugs can be especially difficult due to poor skin permeability. Further, some active agents have poor or low solubility in the adhesive and/or other components used in typical transdermal formulations. Further, there is a need for stable, long term administration of active agents (e.g. 1-10 days or more) that provides a stable and effective release of the agent over the administration period and has suitable adhesion for the long term administration.

Active agents for transdermal delivery are typically provided in their neutral form because the neutral form is typically much more skin permeable than a corresponding salt form. In traditional transdermal formulations, a neutral form of an active agent is solubilized in an adhesive matrix, and the active agent diffuses through the adhesive matrix and into the skin. Transdermal patches, therefore, typically contain as much active agent dissolved in the adhesive matrix as the agent's solubility in the adhesive matrix allows, often with solubilizers to enhance its solubility. Alternatively, neutral, solid particles of active agent are sometimes dispersed in an adhesive matrix, so long as the particles' dissolution rate is such that a constant supply of dissolved active agent is provided.

For many active agents, however, a neutral form is more difficult to solubilize and/or formulate into a composition, system or medicament for administration to a patient or subject. When a drug has a low solubility in an adhesive matrix, as does an unionized neutral form, it is difficult to incorporate a sufficient amount of the drug in a solubilized form in the adhesive in order to deliver at a therapeutic level for multiple days. A further complication is that a dissolved active agent may crystallize within the adhesive matrix during the process of preparing the medicament, e.g., solvation, coating, and drying. Further, many active agents are less stable in neutral form than in salt form. Therefore, there exists a need for compositions, systems and medicaments having an adhesive matrix as a component layer that can consistently and effectively deliver a therapeutic amount of an active agent over a prolonged period of time.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In a first aspect, a transdermal delivery system is provided, the system comprising a skin contact adhesive layer to attach the system to the skin of a user; a drug reservoir layer comprising an active agent and a drug carrier composition; and a microporous membrane disposed between the adhesive layer and the drug reservoir layer, the microporous membrane comprising a plurality of pores and a membrane treatment composition, wherein the membrane treatment composition occupies at least a portion of the pores.

In some embodiments, the microporous membrane is and/or the pores of the microporous membrane are saturated with the membrane treatment composition. In another embodiment, the membrane treatment composition is sequestered in or within the pores of the microporous membrane. In another embodiment, the membrane treatment composition fills the pores of the microporous membrane. In one embodiment, the microporous membrane is a flat sheet microporous membrane.

In some embodiments, the drug carrier composition and the membrane treatment composition are the same. In another embodiment, the drug carrier composition and the membrane treatment composition are different. In one embodiment, the drug carrier composition and the membrane treatment composition are different. In one embodiment, the membrane treatment composition and the contact adhesive layer drug carrier composition are the same, and both are different from the drug carrier composition that is disposed in the drug reservoir layer. In one embodiment, the drug carrier composition differs from the membrane treatment composition and the contact adhesive layer drug carrier composition by the presence of a hydrophilic solvent.

In some embodiments, the membrane treatment composition comprises a nonionic surfactant, a long-chain aliphatic alcohol, a citric acid ester, and/or combinations thereof.

In some embodiments, the active agent is a water insoluble base and the drug carrier composition comprises a nonionic surfactant, a long-chain aliphatic alcohol, a citric acid ester, and/or combinations thereof.

In some embodiments, the microporous membrane is a microporous polypropylene.

In some embodiments, the microporous membrane has pores with an average pore size of from about 0.001 μm to about 100 μm.

In some embodiments, the pore size is from about 0.010 μm to about 0.100 μm.

In some embodiments, the pore size is from about 0.040 μm to about 0.050 μm.

In some embodiments, the microporous membrane has a porosity of about 30% to about 50%.

In some embodiments, the drug reservoir layer additionally comprises glycerine.

In some embodiments, the glycerine is present in the amount of about 5 wt % to about 15 wt %.

In some embodiments, the membrane treatment composition does not include glycerine.

In some embodiments, the drug reservoir layer further comprises a crosslinked polyvinylpyrrolidone.

In some embodiments, the crosslinked polyvinylpyrrolidone is present in the amount of about 10 wt % to about 20 wt %.

In some embodiments, the active agent to be administered to a subject is generated in situ in the drug reservoir layer by reaction of a pharmaceutically acceptable salt of the active agent and an amphoteric base compound.

In some embodiments, the amphoteric inorganic compound in the drug reservoir layer is present in the amount of about 2 wt % to about 5 wt % of the drug reservoir layer.

In some embodiments, the amphoteric inorganic compound in the drug reservoir layer is an alkaline salt.

In some embodiments, the alkaline salt is sodium bicarbonate.

In some embodiments, the active agent to be administered to a subject is donepezil base.

In some embodiments, the pharmaceutically acceptable salt is donepezil hydrochloride.

In some embodiments, the donepezil hydrochloride is present in the drug reservoir layer in the amount of about 5 wt % to about 25 wt % of the drug reservoir layer.

In some embodiments, the drug reservoir layer comprises about 5 wt % to about 15 wt % triethyl citrate.

In some embodiments, the drug reservoir layer comprises about 0.5 wt % to about 5 wt % sorbitan monolaurate.

In some embodiments, the drug reservoir layer comprises about 0.5% to about 5% lauryl lactate.

In some embodiments, the drug reservoir layer comprises about 0.1 wt % to about 2 wt % of ascorbic palmitate.

In some embodiments, the drug reservoir layer comprises about 35 wt % to about 50 wt % of a copolymer of acrylic acid and vinyl acetate.

In some embodiments, the drug carrier composition comprises triethyl citrate, lauryl lactate, sorbitan monolaurate, or combinations thereof.

In some embodiments, the drug carrier composition comprises about 60 wt % to about 75 wt % triethyl citrate.

In some embodiments, the drug carrier composition comprises about 10 wt % to about 17 wt % sorbitan monolaurate.

In some embodiments, the drug carrier composition comprises about 15 wt % to about 25 wt % lauryl lactate.

In some embodiments, the drug carrier composition comprises about 66.7 wt % triethyl citrate; about 20.0 wt % lauryl lactate; and about 13.3 wt % sorbitan monolaurate.

In some embodiments, the drug reservoir layer comprises about 10 wt % to about 20 wt % of the drug carrier composition.

In some embodiments, the drug reservoir layer comprises about 16.0 wt % donepezil hydrochloride; about 2.6 wt % sodium bicarbonate; about 10.0 wt % triethyl citrate; about 3.0 wt % lauryl lactate; about 2.0 wt % sorbitan lacate; about 10.0 wt % glycerine; about 15.0 wt % crosslinked polyvinylpyrrolidone; about 0.5 wt % ascorbic palmitate; and about 40.9 wt % copolymer of acrylic acid and vinyl acetate.

In some embodiments, the membrane treatment composition comprises triethyl citrate, lauryl lactate, sorbitan monolaurate, and/or combinations thereof.

In some embodiments, the membrane treatment composition comprises about 60 wt % to about 75 wt % triethyl citrate.

In some embodiments, the membrane treatment composition comprises about 10 wt % to about 17 wt % sorbitan monolaurate.

In some embodiments, the membrane treatment composition comprises about 15 wt % to about 25 wt % lauryl lactate.

In some embodiments, the membrane treatment composition comprises about 66.7 wt % triethyl citrate; about 20.0 wt % lauryl lactate; and about 13.3 wt % sorbitan monolaurate.

In some embodiments, the system is configured to provide a dose of about 5 mg to about 10 mg of donepezil base per day.

In some embodiments, the skin contact adhesive layer comprises a contact adhesive layer drug carrier composition.

In some embodiments, the contact adhesive layer drug carrier composition comprises triethyl citrate, lauryl lactate, sorbitan monolaurate, and/or combinations thereof. In one embodiment, the contact adhesive layer drug carrier composition comprises about 66.7 wt % triethyl citrate; about 20.0 wt % lauryl lactate; and about 13.3 wt % sorbitan monolaurate.

In some embodiments, the contact adhesive layer drug carrier composition is present in the contact adhesive layer in the amount of about 10 wt % to about 20 wt %.

In some embodiments, the active agent is memantine base.

In some embodiments, the pharmaceutically acceptable salt is memantine hydrochloride.

In some embodiments, the memantine hydrochloride is present in the drug reservoir layer in the amount of about 15 wt % to about 35 wt %.

In some embodiments, the drug carrier composition comprises octyldodecanol.

In some embodiments, the octyldodecanol is present in the amount of about 5 wt % to about 15 wt %.

In some embodiments, the drug reservoir layer comprises about 25 wt % to about 40 wt % of a copolymer of acrylic acid and vinyl acetate.

In some embodiments, the skin contact adhesive layer comprises a hydrophilic fumed silica in the amount of about 5 wt % to about 10 wt %.

In some embodiments, the membrane treatment composition comprises octyldodecanol.

In some embodiments, the system is configured to provide a dose of about 1 mg to about 30 mg of memantine base per day.

In some embodiments, the drug reservoir layer comprises about 25 wt % memantine hydrochloride; about 9.73 wt % sodium bicarbonate; about 7.0 wt % octyldodecanol; about 10.0 wt % glycerine; about 15.0 wt % crosslinked polyvinylpyrrolidone; and about 33.27 wt % copolymer of acrylic acid and vinyl acetate.

In some embodiments, the skin contact adhesive layer comprises about 5 wt % to about 15 wt % octyldodecanol.

In some embodiments, the contact adhesive layer comprises about 10 wt % octyldodecanol.

In some embodiments, the active agent is fingolimod.

In some embodiments, the pharmaceutically acceptable salt is fingolimod hydrochloride.

In some embodiments, the skin contact adhesive layer comprises a copolymer of acrylic acid and vinyl acetate.

In some embodiments, the copolymer of acrylic acid and vinyl acetate is present in the amount of about 60 wt % to about 75 wt %.

In some embodiments, the skin contact adhesive layer comprises a polyisobutylene.

In some embodiments, the polyisobutylene is present in the amount of about 65 wt % to about 90 wt %.

In some embodiments, the skin contact adhesive layer further comprises crosslinked polyvinylpyrrolidone.

In some embodiments, the crosslinked polyvinylpyrrolidone is present in the amount of about 15 wt % to about 25 wt %.

In some embodiments, the transdermal delivery systems described herein can further comprise a first backing layer in contact with the drug reservoir layer; an adhesive overlay in contact with the first backing layer on the side opposite from the drug reservoir layer; and a second backing layer in contact with the adhesive overlay on the side opposite from the first backing layer.

In some embodiments, the first backing layer comprises a polyester laminate.

In some embodiments, the adhesive overlay comprises a polyisobutylene, a polybutene, a crosslinked polyvinylpyrrolidone, an acrylic adhesive, a copolymer of acrylic acid and vinyl acetate, or combinations thereof.

In some embodiments, the adhesive overlay comprises a copolymer of acrylic acid and vinyl acetate.

In some embodiments, the second backing layer comprises a woven polyester fabric.

In some embodiments, the transdermal delivery systems described herein can further comprise a release liner comprising a film, a non-woven fabric, a woven fabric, a laminate, or combinations thereof wherein the release liner is in contact with the skin contact adhesive layer on the opposite side from the intermediate layer.

In some embodiments, the release liner is a silicone-coated polymer film or paper.

In some embodiments, the release liner is a silicone-coated polyethylene terephthalate (PET) film, a fluorocarbon film, or a fluorocarbon coated PET film.

In another aspect, a method for transdermal delivery of an active agent is provided, comprising providing any one of the above described transdermal delivery systems, securing, or instructing to secure, the system to the skin of a user to deliver the active agent from the system to the skin, whereby (i) the time lag for steady state flux is at least about 20% faster compared to a system with no membrane treatment composition in the pores of the microporous membrane, (ii) the system achieves its steady state equilibrium flux at least 20% faster compared to a system with no membrane treatment composition in the pores of the microporous membrane; and/or (iii) the active agent diffuses from the system to the skin at least 20% faster compared to a system with no membrane treatment composition in the pores of the microporous membrane.

In yet another aspect, a method for treating Alzheimer's disease is provided comprising providing any of the transdermal delivery systems comprising an active agent, such as a donepezil base or a memantine base, as described above for administration to the skin of a patient.

In still another aspect, a method for treating Alzheimer's disease, obsessive compulsive disorder, anxiety disorder, attention deficit hyperactivity disorder (ADHD), or opioid dependence is provided comprising providing any of the transdermal delivery systems comprising a memantine compound as described above to the skin of a patient.

In another aspect, a method for manufacturing a transdermal delivery system of an active agent is provided comprising providing a skin contact adhesive layer to attach the system to the skin of a user; providing a drug reservoir layer comprising an active agent and a drug carrier composition; treating a microporous membrane having a plurality of pores with a membrane treatment composition to provide a pretreated microporous membrane, wherein at least a portion of the pores of the pretreated microporous membrane contain the membrane treatment composition; and providing an intermediate layer disposed between the skin contact adhesive layer and the drug reservoir layer, wherein the intermediate layer comprises the pretreated microporous membrane;

In some embodiments of the manufacturing method, the microporous membrane comprises a microporous polypropylene.

In some embodiments of the manufacturing method, the active agent of the drug reservoir layer is generated in situ by reaction of a pharmaceutically acceptable salt of the active agent and an amphoteric base compound.

In some embodiments of the manufacturing method, the microporous membrane has an average pore size of from about 0.001 μm to about 100 μm.

In some embodiments of the manufacturing method, the pore size is from about 0.010 μm to about 0.100 μm.

In some embodiments of the manufacturing method, the pore size is from about 0.040 μm to about 0.050 μm.

In some embodiments of the manufacturing method, the microporous membrane has a porosity of about 30% to about 50%.

In some embodiments of the manufacturing method, the step of treating a microporous membrane with a membrane treatment composition comprises contacting the microporous membrane with the membrane treatment composition, allowing the microporous membrane to become saturated with the membrane treatment composition, and removing any excess membrane treatment composition from the saturated microporous membrane.

In some embodiments of the manufacturing method, the membrane treatment composition comprises a nonionic surfactant, a long-chain aliphatic alcohol, a citric acid ester, or combinations thereof.

In some embodiments of the manufacturing method, the amphoteric inorganic base compound is sodium bicarbonate.

In some embodiments of the manufacturing method, the active agent is donepezil base and the pharmaceutically acceptable salt is donepezil hydrochloride.

In some embodiments of the manufacturing method, the drug carrier composition comprises triethyl citrate, lauryl lactate, sorbitan monolaurate, or any combination thereof.

In some embodiments of the manufacturing method, the drug carrier composition comprises about 66.7 wt % triethyl citrate; about 20.0 wt % lauryl lactate; and about 13.3 wt % sorbitan monolaurate.

In some embodiments of the manufacturing method, the membrane treatment composition comprises about 66.7 wt % triethyl citrate; about 20.0 wt % lauryl lactate; and about 13.3 wt % sorbitan monolaurate.

In some embodiments of the manufacturing method, the active agent is memantine and the pharmaceutically acceptable salt is memantine hydrochloride.

In some embodiments of the manufacturing method, the drug carrier composition and the membrane treatment composition both comprise octyldodecanol.

In some embodiments of the manufacturing method, the active agent is fingolimod base and the pharmaceutically acceptable salt is fingolimod hydrochloride.

In some embodiments, the manufacturing method further comprises the steps of providing a first backing layer in contact with the drug reservoir layer; providing an adhesive overlay in contact with the first backing layer on the side opposite from the drug reservoir layer; and providing a second backing layer in contact with the adhesive overlay on the side opposite from the first backing layer.

### BRIEF DESCRIPTION OF THE DRAWINGS

FIGS. 1A-1D are illustrations of transdermal delivery systems according to several embodiments;

FIG. 2A is a graph of mean plasma concentration of donepezil, in ng/ml, as a function of time, in days, in human subjects treated with a donepezil transdermal delivery system (circles) for 1 week, or with 5 mg of donepezil administered orally on day 1 and on day 7 (triangles);

FIG. 2B is a graph showing the mean plasma concentration of donepezil, in ng/ml, in the 24 hour period after oral administration of a 5 mg donepezil tablet (triangles) and after removal of the donepezil transdermal delivery system (circles);

FIG. 3 is a graph showing the projected mean plasma concentration of donepezil, in ng/ml, over a 28 day (4 week) treatment period with a transdermal delivery system designed to administer 10 mg/day for a week (solid line), with a new patch applied once weekly, and over a 28 day period with a 10 mg daily oral tablet of donepezil (dashed line);

FIG. 4 is a bar graph of the number of subjects in the group treated with the donepezil transdermal delivery system for 1 week and the observed skin irritation subsequent to patch removal, where the open bars indicate no skin irritation and the filled bars indicate mild skin irritation;

FIG. 5A shows the mean plasma concentration of donepezil, in ng/ml, at each day in week 5 of a clinical human study where subjects were treated with donepezil administered transdermally from transdermal patch with a first surface area (solid line) and a second, larger surface area (dashed line) and donepezil administered orally, where the donepezil plasma concentration for patients treated orally is indicated by the thick, bold line at days 6-7, and the dotted line shows the projected daily plasma concentration for oral treatment; and

FIG. 5B is a bar graph showing the number of gastrointestinal related adverse events (nausea, vomiting and diarrhea) reported by subjects in a clinical study, where the subjects were treated as described in FIG. 5A; the bars with dashed fill correspond to subjects treated with the weekly smaller size transdermal patch, the bars with vertical line fill correspond to subjects treated with the weekly larger size transdermal patch, and the bars with horizontal line fill correspond to the subjects treated with oral donepezil.

FIG. 6 is a graph of average skin flux for memantine transdermal delivery devices, in μg/cm2·hr, in vitro as a function of time, in hours, in an in vitro skin permeation test.

FIG. 7 is a graph of the average skin flux of donepezil μg/cm2·hr, in vitro as a function of time, in hours, in an in vitro skin permeation test of a transdermal system comprising a pretreated microporous membrane (Squares) in comparison to the skin flux of donepezil of a transdermal system in which the microporous membrane is untreated (Circles).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “drug” or “active agent” or “therapeutically active agent” are used interchangeably.

An “adhesive matrix” as described herein includes matrices made in one piece, for example, matrices made via solvent casting or extrusion as well as matrices formed in two or more portions that are then pressed or joined together.

“Donepezil” as used herein refers to 2,3-dihydro-5,6-dimethoxy-2-[[1-(phenylmethyl)-4-piperidinyl]methyl]-1H-inden-1-one.

The terms “treatment,” “therapy,” “therapeutic” and the like, as used herein, encompass any course of medical intervention aimed at a pathologic condition, and includes not only permanent cure of a disease, but prevention of disease, control or even steps taken to mitigate a disease or disease symptoms.

The term “skin” as used herein refers to skin or mucosal tissue, including the interior surface of body cavities that have a mucosal lining. The term “skin” should be interpreted as including “mucosal tissue” and vice versa.

The term “therapeutically effective amount” as used herein refers to the amount of an active agent that is nontoxic but sufficient to provide the desired therapeutic effect. The amount that is “effective” will vary from subject to subject, depending on the age and general condition of the individual, the particular active agent or agents, and the like as known to those skilled in the art.

The phrase “pharmaceutically acceptable” is employed herein to refer to those compounds, salts, compositions, dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The terms “transdermal” or “transdermal delivery” as used herein refer to administration of an active agent to a body surface of an individual so that the agent passes through the body surface, e.g., skin, and into the individual's blood stream. The term “transdermal” is intended to include transmucosal administration, i.e., administration of a drug to the mucosal (e.g., sublingual, buccal, vaginal, rectal) surface of an individual so that the agent passes through the mucosal tissue and into the individual's blood stream.

The term “treating” is used herein, for instance, in reference to methods of treating a disorder, such as Alzheimer's disease, and generally includes the administration of a compound or composition which reduces the frequency of, or delays the onset of, symptoms of a medical condition (e.g., Alzheimer's disease) in a subject relative to a subject not receiving the compound or composition. This can include reversing, reducing, or arresting the symptoms, clinical signs, and underlying pathology of a condition in a manner to improve or stabilize a subject's condition (e.g., regression of mental facilities).

The compositions of the present disclosure can comprise, consist essentially of, or consist of, the components disclosed.

All percentages, parts and ratios are based upon the total weight of the topical compositions and all measurements made are at about 25° C., unless otherwise specified.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Transdermal Delivery System and Compositions for Use in a Transdermal Delivery System

A transdermal delivery system for systemic delivery of water-insoluble drug base is provided. The transdermal system in general is comprised of a skin contact adhesive layer and a drug reservoir layer, where the two layers are separated by an intermediate layer that includes a microporous membrane that has been pretreated with a membrane treatment composition. The system can include additional layers as are described below. The composition of the layers in the system are now described.

In some embodiments, the drug reservoir comprises as an active agent a donepezil compound or a derivative thereof. Donepezil is an acetylcholinesterase inhibitor with the chemical structure 2,3-Dihydro-5,6-dimethoxy-2-[[1-(phenylmethyl)-4-piperidinyl]methyl]-1H-inden-1-one:

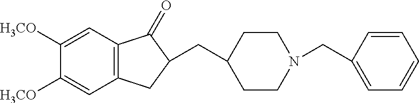 [SMILES: COc1cc2c(cc1OC)C(=O)C(CC1CCN(Cc3ccccc3)CC1)C2]

Donepezil has a molecular weight of 379.5 and is lipophilic (Log P value 3.08-4.11).

In some embodiments, the drug reservoir comprises, as active ingredient, a memantine compound or a derivative thereof. Memantine (NAMENDA) is a compound that belongs to the admantane class of active agents. In some embodiments, the compound comprises the structure shown in Formula I. In another embodiment, the memantine compound is also known as 3,5-dimethyladamantan-1-amine; 1-amino-3,5-dimethyladamantane; 1,3-dimethyl-5-adamantanamine; 3,5-dimethyl-1-adamantanamine; 3,5-dimethyl-1-aminoadamantane; and 3,5-dimethyltricyclo (3.3.1.1 (3,7)) decan-1-amine:

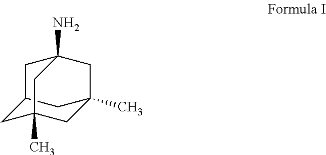

In some embodiments, the drug reservoir layer comprises, as active agent, a fingolimod compound or a derivate thereof.

The drug reservoir layer may additionally include adjunct components conventionally found in pharmaceutical compositions in their art-established fashion and at their art-established levels. F or example, the compositions may contain additional compatible pharmaceutically active materials for combination therapy, e.g., donepezil (ARICEPT®), memantine, rivastigmine (EXCELON®), galantamine (RAZADYNE®), icopezil, pyridostigmine, edrophonium, neostigmine, physostigmine, Huperzine A, phenserine, tacrine, including, L-type calcium channel blocker selected from amlodipine, felodipine, isradipine, lacidipine, lercanidipine, nicardipine, nifedipine, nimodipine, nitrendipine, nisoldipine, or (+) isopropyl 2-methoxyethyl 4-(2-chloro-3-cyano-phenyl)-1,4-dihydro-2,6-dimethylpyridine-3,5-dicarboxylate, or a combination thereof. See, U.S. pat. Pub. No. 2009/0156639.

The drug reservoir layer, in one embodiment, is a composition comprising an adhesive matrix comprising an adhesive polymer, a drug carrier composition and donepezil base generated in situ in the drug reservoir layer after the transdermal system is applied to the skin by reaction of a donepezil salt and an alkaline salt or another amphoteric base compound. The drug reservoir layer is manufactured using a salt form of donepezil, e.g., donepezil hydrochloride (HCl), and an alkaline salt that react in situ to form donepezil base after the transdermal system is applied to the skin. The alkaline salt can be, for example, sodium carbonate, sodium bicarbonate, potassium carbonate, potassium bicarbonate, trisodium phosphate, disodium hydrogen phosphate, sodium oxylate, sodium succinate, sodium citrate, or sodium salicylate.

The drug reservoir also comprises a drug carrier composition. In one embodiment, the drug carrier composition is a solvent composition comprised of one, two, three or four solvents. In one embodiment, the drug carrier composition comprises triethyl citrate; and in other embodiments, one or both of glycerine and sorbitan monolaurate are additionally present. In another embodiment, an α-hydroxy acid as a further solvent in the drug carrier composition is present. Exemplary α-hydroxy acid solvents are esters of lactic acid or glycolic acid, and an example is lauryl lactate. In one embodiment, the drug carrier composition is comprised of, consists essentially of, or consists of triethyl citrate, sorbitan monolaurate, lauryl lactate and glycerine.

The adhesive component in the drug reservoir can be any of a variety of adhesive materials, such as pressure sensitive adhesive polymers. Polyacrylate pressure sensitive adhesive polymers are an example, and typically comprise a polyacrylate that is a polymer or a copolymer of a monomer or monomers selected from acrylic acid esters and methacrylic acid esters. Other monomers, such as acrylic acid and vinyl acetate, may be present. In embodiments, the acrylic polymer is based on acrylic esters such as 2-ethylhexyl acrylate (2-EHA) and ethyl acrylate. In some embodiments, the polyacrylate polymer is a polymer or a copolymer of a monomer or monomers selected from acrylic acid and vinyl acetate. In embodiments, the acrylic polymer adhesive has pendent carboxyl (—COOH) or hydroxyl (—OH) functional groups. In embodiments, the acrylic polymer adhesive comprises at least one of polyacrylate, polymethacrylate, derivatives thereof, and co-polymers thereof. In embodiments, the acrylic adhesive is comprised of an acrylate copolymer comprising acrylic ester monomers, acrylic acid, and/or vinyl acetate monomers. A copolymer of acrylic acid and vinyl acetate is one example. Acrylate copolymers are sold under the trade-name DURO-TAK® and include, but are not limited to, DURO-TAK 387-2516, 387-2051, and 387-2074.

The drug reservoir may also comprise a copolymer such as a polyvinylpyrrolidone/vinyl acetate copolymer, an acrylic acid/vinyl acetate copolymer, or a vinyl acetate/ethylene acetate copolymer. In one embodiment, the copolymer is a vinyl acetate/N-vinylpyrrolidone copolymer such as the copolymer sold as Plasdone™ S630 (Ashland). In another embodiment, the polyvinylpyrrolidone-vinyl acetate copolymer is a linear random copolymer of n-vinyl-2-pyrrolidone and vinyl acetate. In one embodiment, the copolymer is a 60:40 copolymer of n-vinyl-2-pyrrolidone and vinyl acetate.

The drug reservoir may also comprise a polyvinylpyrrolidone (PVP). PVP is a water-soluble polymer comprised of the N-vinylpyrrolidone monomer, and is available in various forms, including cross-linked and non-crosslinked. In some of the working examples herein, a cross-linked PVP is included in the drug reservoir.

In some embodiments, the drug reservoir comprises at least about 25-80 wt % of adhesive polymers relative to the weight of the drug reservoir (inclusive of sub-ranges). In embodiments, the drug reservoir comprises at least about 35-80%, 30-75%, at least about 40-75%, at least about 50-75%, at least about 60-75%, at least about 25-70%, at least about 30-70%, at least about 40-70%, at least about 50-70%, at least about 60-70%, at least about 25-60%, at least about 30-60%, at least about 40-60%, at least about 50-60%, at least about 25-50%, at least about 30-50%, at least about 40-50%, at least about 25-40%, at least about 30-40%, or at least about 25-30% of an adhesive polymer or copolymer or mixture of polymers and/or copolyemrs (all percentages in wt %). It will be appreciated that the drug reservoir adhesive matrix may include one or more or at least one adhesive polymers or copolymers. In embodiments, the drug reservoir comprises at least about 5-75% of an individual polymer relative to the total weight of the polymers in the matrix. In embodiments, the drug reservoir comprises at least about 5-10%, 5-15%, 5-20%, 5-25%, 5-30%, 5-40%, 5-50%, 5-60%, 5-70%, 5-75%, 10-15%, 10-20%, 10-20%, 10-25%, 10-30%, 10-40%, 10-50%, 10-60%, 10-70%, 10-75%, 15-20%, 15-25%, 15-30%, 15-40%, 15-50%, 15-60%, 15-70%, 15-75%, 20-25%, 20-30%, 20-40%, 20-50%, 20-60%, 20-70%, 20-75%, 25-30%, 25-40%, 25-50%, 25-60%, 25-70%, 25-75%, 30-40%, 30-50%, 30-60%, 30-70%, 30-75%, 40-50%, 40-60%, 40-70%, 40-75%, 50-60%, 50-70%, 50-75%, 60-70%, 60-75%, or 70-75% of an individual polymer.

In one exemplary drug reservoir, a matrix that comprises or consists essentially of donepezil base generated in situ by reaction of donepezil HCl and sodium bicarbonate; a drug carrier composition mixture of triethyl citrate, sorbitan monolaurate, and glycerine; and a polymeric, adhesive matrix of crosslinked polyvinylpyrrolidone and a copolymer of acrylic acid/vinyl acetate is contemplated. In another exemplary drug reservoir, a composition, comprising an adhesive matrix that comprises or consisting essentially of donepezil base generated in situ by reaction of between about 10-25 wt % donepezil HCl and between about 1-5 wt % sodium bicarbonate; about 5-15 wt % triethyl citrate; about 0.5-5 wt % sorbitan monolaurate; about 5-15 wt % glycerine; about 5-25 wt % crosslinked polyvinylpyrrolidone; and about 30-50 wt % acrylate-vinylacetate copolymer is contemplated. In another example, a composition comprising an adhesive matrix consisting essentially of donepezil base generated in situ by reaction of between about 14-18 wt % donepezil HCl and between about 2-5 wt % sodium bicarbonate; about 8-12 wt % triethyl citrate; about 1.5-2.5 wt % sorbitan monolaurate; about 9-11 wt % glycerine; about 13-17 wt % crosslinked polyvinylpyrrolidone; and about 40-42 wt % acrylate-vinylacetate copolymer is contemplated.

A drug reservoir as described herein and hereinabove is contemplated for use in a transdermal delivery system, where the system additionally comprises a skin contact adhesive. The skin contact adhesive layer may be fabricated from any of the adhesive materials listed herein and hereinabove. The skin contact adhesive layer, in one embodiment comprises between about 50-90 wt % of adhesive polymer or copolymer, or between about 55-90 wt %, or between about 60-90 wt %, between about 65-90 wt %, between about 70-90 wt %, between about 75-90 wt %, or between about 80-90 wt %. In one embodiment, the skin contact adhesive is comprised of a copolymer of acrylic acid/vinyl acetate. In another embodiment, the skin contact adhesive layer additionally comprises a polyvinylpyrrolidone, such as a crosslinked polyvinylpyrrolidone.

In one embodiment, the skin contact adhesive layer comprises one or more biocompatible polymers selected from one or more of polyisobutylene (PIB), a silicone polymer, acrylate copolymers, butyl rubber, polybutylene, styrene-iosprene-styrene block copolymers, styrene-butadiene-styrene block copolymers, ethylene-vinyl acetate (EVA), mixtures and copolymers thereof. In one embodiment, the biocompatible polymer is polyisobutylene.

In one embodiment, the biocompatible polymer is a PIB-based matrix comprising PIB Oppanol B100 (BASF, MW=1,100,000), PIB Oppanol B 12 (BASF, MW=51,000, MW/MN=3.2) and polybutenc (PB) Indopol H1900 (INEOS oligomers, MW=4500, MW/MN=1.8). The weight ratio between components of the PIB matrix is as follows: PIB Oppanol B100: PIB Oppanol B 12: Indopol H1900=10:50:40 (See, Brantseva et al., European Polymer Journal, 76, 228-244, 2016).

In one embodiment, the skin contact adhesive layer comprises a biocompatible polymer, containing about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.9, or greater % by weight, wherein all values are relative to the weight of the adhesive layer. Particularly, the % weight of the biocompatible polymer in the adhesive layer is between about 50%-95%, especially about 60%-80%, of the entire skin contact adhesive layer. In some embodiments, the amount of the biocompatible polymer in the skin contact adhesive layer is at least about 50-90%, 50-85%, 50-80%, 50-75%, 50-70%, 50-65%, 50-60%, 50-55%, 55-95%, 55-90%, 55-85%, 55-80%, 55-75%, 55-70%, 55-65%, 55-60%, 60-95%, 60-90%, 60-85%, 60-80%, 60-75%, 60-70%, 60-65%, 65-95%, 65-90%, 65-85%, 65-80%, 65-75%, 65-70%, 70-95%, 70-90%, 70-85%, 70-80%, 70-75%, 75-95%, 75-90%, 75-85%, 75-80%, 80-95%, 80-90%, 80-85%, 85-95%, 85-90%, or 90-95%.

The skin contact adhesive layer may also comprise a skin contact adhesive layer drug carrier composition. In embodiments, the skin contact adhesive layer comprises as a contact adhesive layer drug carrier composition one or more of a citric ester, a surfactant and/or an alpha-hydroxy acid. In one embodiment, the skin contact adhesive layer comprises as a contact adhesive layer drug carrier composition one or more of triethyl citrate, sorbitan monolaurate, and/or lauryl lactate. In one embodiment, the skin contact adhesive layer as manufactured does not include a pharmaceutically active agent intended for systemic delivery—for example, the ingredients combined to form the skin contact adhesive layer and/or the contact adhesive layer drug carrier composition do not include a base form or a salt form of a drug, such as donepezil base or a donepezil salt. During use, after the skin contact adhesive layer is applied to the skin of a user, the base form of the active agent that is generated in situ in the drug reservoir partitions into the drug carrier composition in the drug reservoir, then partitions and moves into the membrane treatment composition in the microporous membrane, and then partitions and moves into the contact adhesive layer drug carrier composition for delivery to the skin of the user.

The drug carrier composition in either or both of the skin contact adhesive layer and the drug reservoir adhesive matrix may be chosen from a wide range of such compounds known in the art. In some embodiments, drug carrier composition for use in the adhesive layer or matrix include, but are not limited to, methyl laurate, propylene glycol monolaurate, glycerol monolaurate, glycerol monooleate, lauryl lactate, myristyl lactate, and dodecyl acetate. Additional drug carrier compositions are described in U.S. Pat. No. 8,874,879, which is incorporated herein by reference. It will be appreciated that the compositions herein may include one or more or at least one drug carrier composition. In embodiments, the penetrating or permeating enhancer is included in an amount between about 1-10%, about 2-5%, about 2-10% relative to the weight of the adhesive matrix (inclusive of sub-ranges).

In one embodiment, the contact adhesive layer drug carrier composition and the membrane treatment composition have one, two, or three identical solvents. In one embodiment, the contact adhesive layer drug carrier composition and the membrane treatment composition are comprised of the same solvents. For example, in one embodiment, the contact adhesive layer drug carrier composition and the membrane treatment composition each comprise a citrate ester, a surfactant, and/or an alpha-hydroxy acid. In one embodiment, the drug carrier composition (in the drug reservoir) comprises a hydrophilic solvent that is excluded from, or is not present in, the membrane treatment composition or in the contact adhesive layer drug carrier composition.

Either or both of the skin contact adhesive layer and the drug reservoir adhesive matrix may further include one or more matrix modifiers. Without wishing to be bound by theory, it is believed that the matrix modifier facilitates homogenization of the adhesive matrix. Sorption of hydrophilic moieties is a possible mechanism for this process. Thus, known matrix modifiers which are to some degree water-sorbent may be used. For example, possible matrix modifiers include colloidal silicone dioxide, fumed silica, cross-linked polyvinylpyrrolidone (PVP), soluble PVP, cellulose derivatives (e.g. hydroxypropyl cellulose (HPC), hydroxyethylcellulose (HEC)), polyacrylamide, polyacrylic acid, a polyacrylic acid salt, or a clay such as kaolin or bentonite. An exemplary commercial fumed silica product is Cab-O-Sil (Cabot Corporation, Boston, Mass.). The hydrophilic mixtures described in U.S. Published Patent Application No. 2003/0170308 may also be employed, for example mixtures of PVP and PEG or of PVP, PEG, and a water-swellable polymer such as EUDRAGIT® L100-55. In embodiments, the matrix modifier is individually included in an amount between about 1-25%, about 2-25%, about 5-25%, about 5-7%, about 7-20%, or about 7-25% relative to the weight of the adhesive matrix (inclusive of sub-ranges). In some embodiments, the matrix modifier does not include ethylcellulose.

Either or both of the skin contact adhesive layer and the drug reservoir adhesive matrix may further include other conventional additives such as adhesive agents, antioxidants, crosslinking or curing agents, pH regulators, pigments, dyes, refractive particles, conductive species, antimicrobial agents, opacifiers, gelling agents, viscosity modifiers or thickening agents, stabilizing agents, and the like as known in the art. In those embodiments wherein adhesion needs to be reduced or eliminated, conventional detackifying agents may also be used. Other agents may also be added, such as antimicrobial agents, to prevent spoilage upon storage, i.e., to inhibit growth of microbes such as ycasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof. These additives, and amounts thereof, are selected in such a way that they do not significantly interfere with the desired chemical and physical properties of the adhesive and/or active agent.

Either or both of the skin contact adhesive layer and the drug reservoir adhesive matrix may further may also contain irritation-mitigating additives to minimize or eliminate the possibility of skin irritation and/or skin damage resulting from the drug, the enhancer, or other components of the composition. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; chloroquine; and corticosteriods.

In some embodiments, the skin contact adhesive layer optionally comprises highly dispersive silica, e.g., hydrophobic colloidal silica that can effectively adsorb hydrophobic drugs and other hydrophobic ingredients. By using hydrophobic colloidal silica at a certain percentage as an excipient (from about 3% to about 20%, preferably from about 5% to about 10% in the formulation), the diffusion of the active ingredient through the matrix can be controlled during storage. Examples of the dispersive silica for use in the compositions include, but are not limited to, the high purity amorphous anhydrous colloidal silicon dioxide for use in pharmaceutical products sold under the name AEROSIL, e.g., AEROSIL®90, AEROSIL®130, AEROSIL®150, AEROSIL®200, AEROSIL®300, AEROSIL®380, AEROSIL®OX50, AEROSIL®TT600, AEROSIL®MOX80, AEROSIL®COK84, AEROSIL®R202, AEROSIL®R805, AEROSIL®R812, AEROSIL®812S, AEROSIL®R972, and/or AEROSIL® R974 or any other highly disperse silica, especially AEROSIL®200 and/or AEROSIL®R972 can be used as highly disperse silica.

In one embodiment, the skin contact adhesive layer comprises highly dispersive silica at least about 40% by weight relative to the weight of the entire adhesive layer, including, at least about 1% by weight relative to the weight of the adhesive layer, including, at least about 3%, e.g., about 4%, about 5%, about 6%, about 7%, about 8%, about 9%, about 10%, about 11%, about 12%, about 13%, about 14%, about 15%, about 16%, about 17%, about 18%, about 19%, about 20%, or greater % by weight, wherein all values are relative to the weight of the entire adhesive layer.

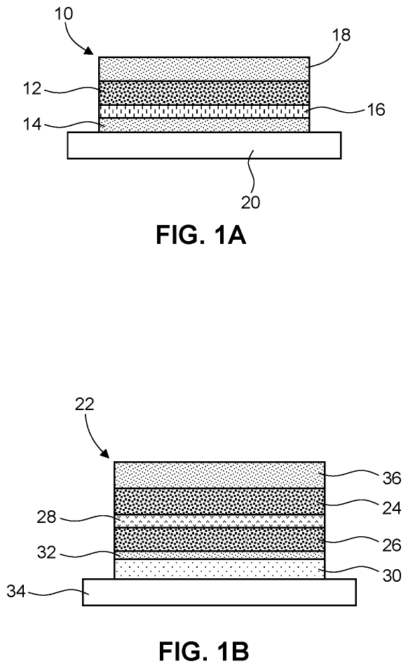

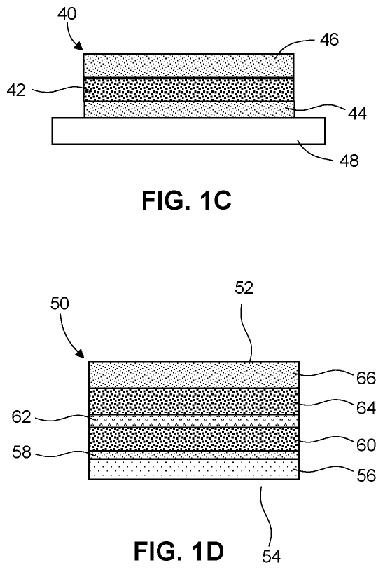

A transdermal delivery system comprised of a drug reservoir adhesive matrix and a skin contact adhesive can have a variety of configurations, and several non-limiting examples are depicted in are set forth in FIGS. 1A-1D. FIG. 1A illustrates a transdermal delivery system 10 comprised of a drug reservoir 12 and a contact adhesive 14 separated by a microporous membrane or by a non-rate controlling material, such as a tic layer composed of a non-woven polyester or polypropylene, 16. A backing layer 18 and a release liner 20 are also present. FIG. 1B illustrates a second embodiment of a transdermal delivery system 22 comprised of a first drug reservoir 24 and a second drug reservoir 26, the first and second drug reservoirs separated by a non-rate controlling material, such as a tic layer composed of a non-woven polyester or polypropylene, 28. A contact adhesive layer 30 provides for attachment of the system to the skin of a user, where a rate controlling membrane 32 controls release of therapeutic agent from the second drug reservoir into the contact adhesive and ultimately onto the skin of a user. A release liner 34 and a backing layer 36 are also present. FIG. 1C shows another embodiment of a transdermal delivery system 40 comprised of a drug reservoir 42 and a contact adhesive layer 44 that provides for attachment of the system to the skin of a user. A backing layer 46 and a release liner 48 are also present.

FIG. 1D shows another embodiment of a transdermal delivery system for systemic delivery of an active agent. The system 50 comprises, in series from the skin facing side 52 to the external environment facing side 54, a skin contact adhesive layer 56 to attach the system to the skin of a user. In one embodiment, the skin contact adhesive layer manufactured is manufactured from an adhesive formulation that does not comprise the active agent or a salt thereof. However, after storage and/or during use, the skin contact adhesive layer comprises the base form of the active agent due to diffusion of base form of the active agent from the drug reservoir layer. Directly in contact with the skin contact adhesive layer is an intermediate layer 58. The intermediate layer can be, for example, a non-woven polyester material or a drug rate-controlling membrane, such as a microporous polyethylene or polyprolylene. The intermediate layer has opposing sides, a skin-facing side (that is in contact with the skin contact adhesive layer 56) and an environment facing side. On the environment facing side of the intermediate layer is a drug reservoir layer 60. The drug reservoir layer is manufactured with an adhesive material, a pharmaceutically acceptable salt of the active agent, and an alkaline salt. The latter two components react in situ to generate the base form of the active agent in the drug reservoir layer that is delivered to the user after application of the system to the skin. In contact with the drug reservoir layer is a first backing layer 62, and in contact with the first backing layer is an adhesive overlay 64. A second backing layer 66 is in contact with the adhesive overlay and with the environment. In one embodiment, the adhesive overlay 64 is composed of two different adhesive layers—for example a first layer of polyisobutylene and polybutene, with or without a crosslinked polyvinylpyrrolidone, and a second layer of an acrylic adhesive.

Accordingly, in one embodiment a transdermal delivery system for systemic delivery of an active agent is provided. The system comprises, in series from the skin facing side to the external environment, a skin contact adhesive layer to attach the system to the skin of a user, the skin contact adhesive layer optionally manufactured from an adhesive formulation that does not comprise the active agent or a salt thereof. Directly in contact with the skin contact adhesive layer is an intermediate layer. On the opposing surface of the intermediate layer is a drug reservoir layer comprised of (i) optionally, a copolymer of acrylic acid/vinyl acetate, (ii) a drug carrier composition as described herein, and (iii) an active agent generated in situ by reaction of a hydrochloride salt of the active agent and an alkaline salt. In contact with the drug reservoir layer is a first backing layer, and in contact with the first backing layer is an adhesive overlay. A second backing layer is in contact with the adhesive overlay and with the environment.

The intermediate layer, also referred to as a fabric layer, a membrane or a tie layer, may be formed of any suitable material including, but not limited to, polyesters, vinyl acetate polymers and copolymers, polyethylenes, and combinations thereof. In one embodiment, the intermediate layer is a nonwoven layer of polyester fibers such as the film sold under the name Recmay® (Kavon Filter Products Co.). In some embodiments, the intermediate layer does not affect the rate of release of the active agent from the adhesive layers.

In some embodiments, the intermediate layer comprises a microporous membrane. For example, the microporous membrane can be a microporous polypropylene or polyethylene. The microporous membrane can help to control the rate of drug release from the transdermal delivery system. Several different microporous membranes are commercially available such as those sold under the name Celgard®, for example the Celgard® 2400 (Polypore International, LP).

Other materials useful in forming the microporous membrane include, but are not limited to polycarbonates, i.e., linear polyesters of carbonic acids in which carbonate groups recur in the polymer chain, by phosgenation of a dihydroxy aromatic such as bisphenol; polyvinylchlorides; polyamides such as polyhexamethylene adipamide and other such polyamides popularly known as nylon; modacrylic copolymers, such as styrene-acrylic acid copolymers; polysulfones such as those of the type characterized by diphenylene sulfone groups in the linear chain thereof are useful; halogenated polymers such as polyvinylidene fluoride, polyvinylfluoride, and polyfluorohalocarbons; polychloroethers and other such thermoplastic polyethers; acetal polymers such as polyformaldehydes; acrylic resins such as polyacrylonitrile polymethyl poly (vinyl alcohol), derivatives of polystyrene such as poly (sodium styrenesulfonate) and polyvinylbenzyltrimethyl-ammonium chloride), poly (hydroxyethyl methacrylate poly(isobutyl vinyl ether); and a large number of copolymers which can be formed by reacting various proportions of monomers from the aforesaid list of polymers are also useful for preparing rate controlling structures useful in the invention.

Diffusion of an active agent through microporous polymeric materials such as microporous polypropylene can be difficult. The polymers are impermeable to the active drugs except at the pore channels, and even then the active agent cannot diffuse through the pores unless it does so in a vaporized state. Thus, if a microporous membrane is used as purchased in the fabrication of a transdermal delivery system, an excessive amount of time may be required for a delivery vehicle (i.e., drug carrier composition) from a drug reservoir layer to partition into the pores and then for the active agent to partition into the delivery vehicle within the pores. The resultant effect is that it can take a long time for the active agent to reach its intended target.

The release rate of an active agent through a microporous membrane can be greatly improved when the microporous membrane is pretreated with a suitable delivery vehicle or membrane treatment composition. Pretreated as used herein intend that the microporous membrane is exposed to a membrane treatment composition to fill pores within the microporous membrane prior to the microporous membrane's incorporation into a transdermal system. The pores of the microporous membrane are filled with or contain a membrane treatment composition prior to and at the time the microporous membrane is incorporated into the transdermal system. The release rate of an active agent through a microporous membrane depends on several variables such as the diffusivity and solubility of the active agent in the membrane treatment composition and the thickness and porosity of the microporous material. For flow of the active agent through the pores of the microporous membrane the concentration gradient, the thickness of the membrane, the viscosity of the active agent, the size of the active agent molecule relative to the pore size, the absolute value of the pore size, and the number of pores or percent voids (porosity) in the material are contributing factors governing solubility and diffusivity of an agent into and through the membrane.

In some embodiments, the microporous membrane can have a porosity in the range of about 30% to about 50%, about 35% to about 45%, or about 40% to about 42%. For example, the microporous membrane can have a porosity of about 30%, 31%, 32%, 33%, 34%, 35%, 36%, 37%, 38%, 39%, 40%, 41%, 42%, 43%, 44%, 45%, 46%, 47%, 48%, 49%, or 50%.

In some embodiments, the microporous membrane can have an average pore size in the range of about 0.001 μm to about 100 μm, about 1 μm to about 10 μm, about 0.010 μm to about 0.100 μm, or about 0.040 μm to about 0.050 μm. For example, the average pore size can be about 0.035 μm, 0.036 μm, 0.037 μm, 0.038 μm, 0.039 μm, 0.040 μm, 0.041 μm, 0.042 μm, 0.043 μm, 0.044 μm, 0.045 μm, 0.046 μm, 0.047 μm, 0.048 μm, 0.049 μm, or 0.050 μm. In some embodiments, the microporous membrane has an average pore size of about 0.043 μm.

The microporous membrane can be pretreated with the same or a different vehicle or membrane treatment composition than the vehicle or drug carrier composition present in the drug reservoir layer. In some embodiments, the microporous membrane is pretreated with a membrane treatment composition comprising a solvent, a surfactant, an emulsifier, a viscosity increasing agent, a stabilizer, a plasticizer, and/or combinations thereof. In some embodiments, the membrane treatment composition does not include a solvent. In some embodiments, the surfactant is a nonionic surfactant. In some embodiments, the microporous membrane is pretreated with a citrate ester. In some embodiments, the citrate ester is triethyl citrate. In some embodiments, the microporous membrane is pretreated with lauryl lactate. In some embodiments, the microporous membrane is pretreated with a sorbitan monoester. In some embodiments, the sorbitan monoester is sorbitan monolaurate (sorbitan laurate). In some embodiments, the microporous membrane is pretreated with a membrane treatment composition comprising triethyl citrate, lauryl lactate, and sorbitan monolaurate. In some embodiments, the microporous membrane is pretreated with octyldodecanol.

In one embodiment, the microporous membrane has a plurality of pores that are filled with or that contain a membrane treatment composition that is different from the drug carrier composition in the drug reservoir layer in fluid communication with the microporous membrane. In one embodiment, the membrane treatment composition does not include (i.e., excludes) a solvent in which the salt form of the active agent is soluble. In one embodiment, the membrane treatment composition does not include (i.e., excludes) a hydrophilic solvent in which the salt form of the active agent is soluble. In one embodiment, the membrane treatment composition does not include (i.e., excludes) a polyol, including solvent polyols, such as polyethylene glycol, propylene glycol, glycerin (glycol), acetonitrile, 1-propanol, N,N-dimethylformamide and dimethyl sulfoxide.

In some embodiments, the contact adhesive layer and/or the drug carrier composition can include a hydrophilic material or component that is not included in the membrane treatment composition. In one embodiment, the hydrophilic material that is present in one or both of the contact adhesive layer and/or the drug carrier composition but is not present in the membrane treatment composition is a hydrophilic solvent such as, but are not limited to, glycerine, water, and mixtures thereof. Other hydrophilic materials include, but are not limited to propylene glycols and low-weight polyethylene glycols. In one embodiment, the microporous membrane is a manufactured from a hydrophobic material to provide a hydrophobic microporous membrane; an example is a polypropylene microporous membrane or a polyethylene microporous membrane. A hydrophilic material, such as a hydrophilic solvent in the drug carrier composition that is within the drug reservoir does not diffuse or permeate into the microporous membrane or into the pores of the microporous membrane due to the hydrophobicity of the membrane material. The hydrophilic material in the drug carrier composition within the drug reservoir layer facilitates and supports the in situ formation of the water insoluble basic active agent from a pharmaceutically acceptable salt thereof. After the base form of the active agent is formed in the drug reservoir layer, the base form of the active agent is solubilized by at least one component in the drug carrier composition and by at least one component in the membrane treatment composition, so that the base form of the active agent diffuses from the drug reservoir layer into and through the hydrophobic pores of the microporous membrane. In one embodiment, the drug carrier composition and the membrane treatment composition have one, two, or three identical solvents, yet the drug carrier composition and the membrane treatment composition are different. For example, in one embodiment, the drug carrier composition and the membrane treatment composition each comprise a citrate ester, a surfactant, and/or an alpha-hydroxy acid, and the drug carrier composition comprises a hydrophilic solvent that is excluded from, or is not present in, the membrane treatment composition.

The drug carrier composition (i) enables the salt form of the active agent to be dissolved and/or suspended in the drug reservoir layer, (ii) supports the in situ reaction of the salt form of the active agent to the base form of the active agent, and (iii) enables the base form of the active agent to be dissolved or solubilized in the drug reservoir, for diffusion into the microporous membrane and into the contact adhesive layer.

The membrane treatment composition enables the base form of the active agent to be dissolved or suspended therein and move diffusionally into and through the microporous membrane. The membrane treatment composition can be either of a liquid or solid nature and can be a poor or good solvent system for the base form of the drug. A membrane treatment composition with poor solvent properties for the base form of the drug is desired when a slow or low rate of release from the transdermal system is desired, and of course the converse is true when the desired release rate is high.

The materials selected for the membrane treatment composition must be non-toxic and those in which the rate controlling microporous material has the required solubility. In another embodiment, the membrane treatment composition is not a solvent for the material from which the microporous membrane is manufactured. That is, the microporous membrane is chemically stable in the membrane treatment composition. The materials which are useful for impregnating, filling, or saturating the pores or micropores of the microporous membrane can be polar, semi-polar or non-polar. Materials for use in a membrane treatment composition in addition to those listed above include, but are not limited to, pharmaceutically acceptable alcohols containing 6 to 25 carbon atoms, such as hexanol, cyclohexanol, benzylalcohol, 1,2-butanediol, glycerol, and amyl alcohol, and octyldodecanol; hydrocarbons having 5 to 12 carbon atoms such as n-hexane, cyclohexane, and ethyl benzene; aldehydes and ketones having 4 to 10 carbon atoms such as heptyl aldehyde, cyclohexanone, and benzaldehyde; esters having 4 to 10 carbon atoms such as amyl acetate and benzyl propionate; etheral oils such as oil of eucalyptus, oil of rue, cumin oil, limonene, thyme], and l-pinene; halogenated hydrocarbons having 2 to 8 carbon atoms such as n-hexyl chloride, n-hexyl bromide, and cyclohexyl chloride; or mixtures of any of the foregoing materials.

In some embodiments, the membrane treatment composition comprises about 60 wt % to about 75 wt % triethyl citrate. In some embodiments, the membrane treatment composition comprises about 55 wt % to about 80 wt %, about 60 wt % to about 70 wt %, about 65 wt % to about 75 wt %, or about 65 wt % to about 70 wt % triethyl citrate. In some embodiments, the membrane treatment composition comprises about 10 wt % to about 17 wt % sorbitan monolaurate. In some embodiments, the membrane treatment composition comprises about 8 wt % to about 25 wt %, about 10 wt % to about 25 wt %, about 8 wt % to about 17 wt %, about 12 wt % to about 20 wt %, about 10 wt % to about 15 wt %, or about 12 wt % to about 14 wt % of sorbitan monolaurate. In some embodiments, the membrane treatment composition comprises about 15 wt % to about 25 wt % lauryl lactate. In some embodiments, the membrane treatment composition can comprise about 10 wt % to about 30 wt %, about 15 wt % to about 30 wt %, about 15 wt % to about 20 wt %, about 10 wt % to about 25 wt %, about 10 wt % to about 20 wt %, about 17 wt % to about 23 wt %, about 18 wt % to about 22 wt %, or about 19 wt % to about 21 wt % of lauryl lactate. In some embodiments, the membrane treatment composition can be formulated with the combination of triethyl citrate, lauryl lactate, and sorbitan monolaurate in any of the ranges recited above. In some embodiments, the membrane treatment composition comprises about 66.7 wt % triethyl citrate; about 20.0 wt % lauryl lactate; and about 13.3 wt % sorbitan monolaurate.

The thickness of the microporous membrane can vary depending on the type of material and the desired characteristics of the microporous membrane (e.g., porosity, micropore size, time diffusion of the active agent through the membrane). In some embodiments, the microporous membrane has a thickness of between about 5 to about 200 μm. In some embodiments, the microporous membrane has a thickness of between about 10 to about 150 μm, about 10 to about 125 μm, about 10 to about 100 μm, about 10 to about 75 μm, about 10 to about 50 μm, about 5 to about 45 μm, about 5 to about 30 μm, about 10 to about 30 μm, about 15 to about 30 μm, or about 20 to about 30 μm. In some embodiments, the microporous membrane has a thickness of about 22 to about 28 μm. In some embodiments, the microporous membrane has a thickness of about 24 to about 26 μm. In some embodiments, the microporous membrane, has a thickness of about 25 μm. It will be appreciated that the thickness provided here is merely exemplary and the actual thickness may be thinner or thicker as needed for a specific formulation

The microporous membrane can be pretreated in a variety of ways. In general, pretreating comprises contacting the microporous membrane with the membrane treatment composition in a sufficient manner and for a sufficient amount of time. In some embodiments, the pretreating of the microporous membrane comprises contacting the microporous membrane with the membrane treatment composition, allowing the microporous membrane to become saturated with the membrane treatment composition, and removing any excess membrane treatment composition from the saturated microporous membrane. In some embodiments, the microporous membrane is soaked in the membrane treatment composition. In some embodiments, the microporous membrane is immersed into a bath of the membrane treatment composition. In some embodiments, the membrane treatment composition is spread onto the microporous membrane until the microporous membrane is saturated and then the excess membrane treatment composition is removed.

The pretreatment of the microporous membrane with the membrane treatment composition can vary in degree. In some embodiments, a portion of the pores of the microporous membrane contain the membrane treatment composition therein. In some embodiments, about one third, about one half, about two thirds, or about three fourths of the pores will contain the membrane treatment composition. In some embodiments, all of the pores will contain the membrane treatment composition. In some embodiments, the portion of the pores containing membrane treatment composition will only be partially filled. In some embodiments, the membrane treatment composition will occupy about one fourth, about one third, about one half, about two thirds, or about three fourths of the space within the occupied pores. In some embodiments, all of the pores of the microporous membrane will be completely filled with the membrane treatment composition and the microporous membrane will thus be saturated with the membrane treatment composition.

The transdermal delivery system can include an adhesive overlay. The adhesive overlay in the delivery system of FIG. 1D is comprised, in one embodiment, of a polyisobutylene and polybutene mixture. In another embodiment, the adhesive overlay is comprised of a first layer and a second layer, the first layer composed of a polyisobutylene, polybutene and crosslinked polyvinylpyrrolidone mixture and the second layer composed of an acrylic adhesive. Polyisobutylene is a vinyl polymer comprised of the isobutylene monomer. In one embodiment, the biocompatible polymer is a PIB-based matrix comprising PIB Oppanol B100 (BASF, MW=1,100,000), PIB Oppanol B 12 (BASF, MW=51,000, MW/MN=3.2) and polybutene (PB) Indopol H1900 (INEOS oligomers, MW=4500, MW/MN=1.8). The weight ratio between components of the PIB matrix is as follows: PIB Oppanol B100: PIB Oppanol B 12: Indopol H1900=10:50:40 (See, Brantseva et al., European Polymer Journal, 76, 228-244, 2016). Polybutene is a viscous, non-drying, liquid polymer, prepared by copolymerization of 1- and 2-butene with a small quantity of isobutylene. In some embodiments, the polybutene in one embodiment has a molecular weight of between about 750-6000 Daltons, preferably between about 900-4000 Daltons, and preferably between about 900-3000 Daltons. In some embodiments the mixture comprises polybutene in the polyisobutylene blend at about 40 weight percent. More generally, the polybutene is present in the polyisobutylene blend in an amount between 20-50 weight percent, or between 25-45 weight percent.

The transdermal delivery system can comprise a backing layer that provides a structural element for holding or supporting the underlying adhesive layer(s). The backing layer may be formed of any suitable material as known in the art. In some embodiments, the backing layer is occlusive. In some embodiments, the backing is preferably impermeable or substantially impermeable to moisture. In one exemplary embodiment, the barrier layer has a moisture vapor transmission rate of less than about 50 g/m²-day. In some embodiments, the backing layer is preferably inert and/or does not absorb components of the adhesive layer, including the active agent. In some embodiments, the backing layer preferably prevents release of components of the adhesive layer through the backing layer. The backing layer may be flexible or nonflexible. The backing layer is preferably at least partially flexible such that the backing layer is able to conform at least partially to the shape of the skin where the patch is applied. In some embodiments, the backing layer is flexible such that the backing layer conforms to the shape of the skin where the patch is applied. In some embodiments, the backing layer is sufficiently flexible to maintain contact at the application site with movement, e.g. skin movement. Typically, the material used for the backing layer should permit the device to follow the contours of the skin or other application site and be worn comfortably on areas of skin such as at joints or other points of flexure, that are normally subjected to mechanical strain with little or no likelihood of the device disengaging from the skin due to differences in the flexibility or resiliency of the skin and the device.

In some embodiments, the backing layer is formed of one or more of a film, non-woven fabric, woven fabric, laminate, and combinations thereof. In some embodiments, the film is a polymer film comprised of one or more polymers. Suitable polymers are known in the art and include elastomers, polyesters, polyethylene, polypropylene, polyurethanes and polyether amides. In some embodiments, the backing layer is formed of one or more of polyethylene terephthalate, various nylons, polypropylene, metalized polyester films, polyvinylidene chloride, and aluminum foil. In some embodiments, the backing layer is a fabric formed of one or more of polyesters such as polyethylene terephthalate, polyurethane, polyvinyl acetate, polyvinylidene chloride and polyethylene. In one particular, but non-limiting embodiment, the backing layer is formed of a polyester film laminate. One particular polyester film laminate is the polyethylene and polyester laminate such as the laminate sold under the name SCOTCHPAK™ #9723.

In embodiments, the device includes a release liner at least partially in contact at least with the adhesive layer to protect the adhesive layer prior to application. The release liner is typically a disposable layer that is removed prior to application of the device to the treatment site. In some embodiments, the release liner preferably does not absorb components of the adhesive layer, including the active agent. In some embodiments, the release liner is impermeable to components of the adhesive layer (including the active agent) and prevents release of components of the adhesive layer through the release liner. In some embodiments, the release liner is formed of one or more of a film, non-woven fabric, woven fabric, laminate, and combinations thereof. In some embodiments, the release liner is a silicone-coated polymer film or paper. In some non-limiting embodiments, the release liner is a silicone-coated polyethylene terephthalate (PET) film, a fluorocarbon film, or a fluorocarbon coated PET film.

The thickness and/or size of the device and/or adhesive matrices may be determined by one skilled in the art based at least on considerations of wearability and/or required dose. It will be appreciated that the administration site for the device will affect the wearability considerations due to the available size of the administration site and the use of the administration site (e.g. need for flexibility to support movement). In some embodiments, the device and/or adhesive matrix has a thickness of between about 25-500 μm. In some embodiments, the device and/or adhesive matrix has a thickness of between about 50-500 μm. In some embodiments, the patch has a size in the range of about 16 cm²-225 cm². It will be appreciated that the thickness and size provided here are merely exemplary and the actual thickness and or size may be thinner/smaller or thicker/larger as needed for a specific formulation.

Fabrication of a transdermal delivery system is routinely done by skilled artisans and involves casting or extruding each of the adhesive layers onto a suitable film such as a release liner or onto another layer of the transdermal delivery system, and drying if needed to remove solvents and/or volatile compounds. Layers of the transdermal delivery system can be laminated together to form the final system.

Transdermal delivery systems and drug reservoir adhesive matrices were prepared to illustrate the embodiments described herein. Examples 1-9 set forth exemplary compositions and delivery systems. As described in Example 1, a transdermal delivery system comprised a drug reservoir and a contact adhesive with a rate controlling membrane situated between the drug reservoir and the contact adhesive, as depicted in FIG. 1A. A drug reservoir in the form of a solid monolithic adhesive reservoir was prepared using an acrylic acid/vinyl acetate copolymer adhesive with drug carrier composition-triethyl citrate, lauryl lactate and ethyl acetate. The drug reservoir contained approximately 5 wt % donepezil hydrochloride and sodium bicarbonate, to generate in situ donepezil base. A contact adhesive layer comprised of the same acrylic acid/vinyl acetate copolymer adhesive, along with triethyl citrate, lauryl lactate and ethyl acetate as drug carrier composition was prepared. A rate controlling membrane, to control the diffusional release of donepezil base from the drug reservoir, separated the drug reservoir and the contact adhesive.

### III. Methods of Treatment

A method for delivering a therapeutic agent transdermally to a subject is provided. In embodiments, the method comprises treatment of one or more central nervous system (CNS) disorders using delivery systems described herein. Examples of CNS disorders include, but are not limited to, dementia (e.g., Alzheimer's disease, Parkinson's disease, Picks disease, fronto-temporal dementia, vascular dementia, normal pressure hydrocephalus, Huntington's disease (HD), and mild cognitive impairment (MCI)), neuro-related conditions, dementia-related conditions, such as epilepsy, seizure disorders, acute pain, chronic pain, chronic neuropathic pain may be treated using the systems and methods described herein. Epileptic conditions include complex partial, simple partial, partials with secondary generalization, generalized-including absence, grand mal (tonic clonic), tonic, atonic, myoclonic, neonatal, and infantile spasms. Additional specific epilepsy syndromes are juvenile myoclonic epilepsy, Lennox-Gastaut, mesial temporal lobe epilepsy, nocturnal frontal lobe epilepsy, progressive epilepsy with mental retardation, and progressive myoclonic epilepsy. The systems and methods described herein are also useful for the treatment and prevention of pain caused by disorders including cerebrovascular disease, motor neuron diseases (e.g. amyotrophic lateral sclerosis (ALS), Spinal motor atrophies, Tay-Sach's, Sandoff disease, familial spastic paraplegia), neurodegenerative diseases (e.g., familial Alzheimer's disease, prion-related diseases, cerebellar ataxia, Friedrich's ataxia, SCA, Wilson's disease, retinitis pigmentosa (RP), ALS, Adrenoleukodystrophy, Menke's Sx, cerebral autosomal dominant arteriopathy with subcortical infarcts (CADASIL); spinal muscular atrophy, familial ALS, muscular dystrophies, Charcot Maric Tooth diseases, neurofibromatosis, von-Hippel Lindau, Fragile X, spastic paraplesia, psychiatric disorders (e.g., panic syndrome, general anxiety disorder, phobic syndromes of all types, mania, manic depressive illness, hypomania, unipolar depression, depression, stress disorders, posttraumatic stress disorder (PTSD), somatoform disorders, personality disorders, psychosis, and schizophrenia), and drug dependence (e.g., alcohol, psychostimulants (e.g., crack, cocaine, speed, meth), opioids, and nicotine), Tuberous sclerosis, and Wardenburg syndrome), strokes (e.g., thrombotic, embolic, thromboembolic, hemorrhagic, venoconstrictive, and venous), movement disorders (e.g., Parkinson's disorder (PD), dystonias, benign essential tremor, tardive dystonia, tardive dyskinesia, and Tourette's syndrome), ataxic syndromes, disorders of the sympathetic nervous system (e.g., Shy Drager, Olivopontoicerebellar degeneration, striatonigral degeneration, Parkinson's disease (PD), Huntington's disease (HD), Gullian Barre, causalgia, complex regional pain syndrome types I and II, diabetic neuropathy, and alcoholic neuropathy), Cranial nerve disorders (e.g., Trigeminal neuropathy, trigeminal neuralgia, Menier's syndrome, glossopharangela neuralgia, dysphagia, dysphonia, and cranial nerve palsics), myclopethics, traumatic brain and spinal cord injury, radiation brain injury, multiple sclerosis, Post-meningitis syndrome, prion diseases, myelities, radiculitis, neuropathies (e.g., Guillian-Barre, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathics, leprous neuropathy, Bell's palsy, compression neuropathies, sarcoidosis-induced neuropathy, polyneuritis cranialis, heavy metal induced neuropathy, transition metal-induced neuropathy, drug-induced neuropathy), axonic brain damage, encephalopathies, and chronic fatigue syndrome. The systems and methods described herein are also useful for the treatment multiple sclerosis, in particular relapsing-remitting multiple sclerosis, and prevention of relapses in multiple sclerosis and/or in relapsing-remitting multiple sclerosis. All of the above disorders may be treated with the systems and methods described herein.

In embodiments, compositions and devices comprising donepezil are useful for treating, delaying progression, delaying onset, slowing progression, preventing, providing remission, and improvement in symptoms of cognitive disorders or disease are provided herein. In embodiments, compositions and devices comprising donepezil are provided for maintaining mental function including, but not limited to a least one of maintaining thinking, memory, speaking skills as well as managing or moderating one or more behavioral symptoms of a cognitive disorder or disease. In embodiments, the cognitive disorder is Alzheimer's disease. In particular embodiments, the cognitive disorder is Alzheimer's type dementia. In embodiments, compositions and devices comprising donepezil are provided for use in treating, etc. mild, moderate, or severe Alzheimer's disease.

The terms “treatment,” “therapy,” “therapeutic” and the like, as used herein, encompass any course of medical intervention aimed at a pathologic condition, and includes not only permanent cure of a disease, but prevention of disease, control or even steps taken to mitigate a disease or disease symptoms. For instance, in reference to methods of treating a disorder, such as Alzheimer's disease, the embodiment, generally includes the administration of an active agent which reduces the frequency of, or delays the onset of, symptoms of the medical condition in a subject relative to a subject not receiving the active agent. This can include reversing, reducing, or arresting the symptoms, clinical signs, and underlying pathology of a condition in a manner to improve or stabilize a subject's condition (e.g., regression of mental facilities).

In one embodiment, the therapeutic embodiments are carried out by contacting a tissue of a subject, e.g., skin tissue, with the transdermal delivery systems provided herein.

In another embodiment, the therapeutic embodiments are carried out by transdermally administering the active agent to a subject, e.g., a patient suffering from a CNS disorder such as Alzheimer's disease and/or dementia. The term “administering” means applying as a remedy, such as by the placement of an active agent in a manner in which such drug would be received, e.g., transdermally, and be effective in carrying out its intended purpose.

A “subject” or “patient” in whom administration of the therapeutic agent is an effective therapeutic regimen for a disease or disorder is preferably a human, but can be any animal, including a laboratory animal in the context of a trial or screening or activity experiment. Thus, as can be readily appreciated by one of ordinary skill in the art, the methods and systems as provided herein are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use.

Treatment of a subject with the systems may be monitored using methods known in the art. See, e.g., Forchetti et al., “Treating Patients with Moderate to Severe Alzheimer's Disease: Implications of Recent Pharmacologic Studies.” Prim Care Companion J Clin Psychiatry, 7 (4): 155-161, 2005 (PMID: 16163398). The efficacy of treatment using the system is preferably evaluated by examining the subject's symptoms in a quantitative way, e.g., by noting a decrease in the frequency of adverse symptoms, behaviors, or attacks, or an increase in the time for sustained worsening of symptoms. In a successful treatment, the subject's status will have improved (i.e., frequency of relapses will have decreased, or the time to sustained progression will have increased).

Based on the exemplary transdermal delivery systems (also referred to as transdermal devices or devices) described herein, a method for treating a suitable condition with an active agent is provided. In embodiments, devices comprising the active agent are useful for treating, delaying progression, delaying onset, slowing progression, preventing, providing remission, and improvement in symptoms of cognitive disorders or disease and of multiple sclerosis are provided herein. In embodiments, devices comprising the active agent are provided for maintaining mental function including, but not limited to a least one of maintaining thinking, memory, speaking skills as well as managing or moderating one or more behavioral symptoms of a cognitive disorder or disease. In embodiments, the cognitive disorder is Alzheimer's disease. In particular embodiments, the cognitive disorder is Alzheimer's type dementia. In embodiments, devices comprising memantine are provided for use in treating, etc. mild, moderate, or severe Alzheimer's disease. In other embodiments, devices comprising fingolimod are provided for use in treating multiple sclerosis, preventing and/or reducing frequency of relapses of multiple sclerosis, in particular of relapsing-remitting multiple sclerosis.

In one embodiment, the methods relate to therapy of CNS disorders or of autoimmune disorders in a subject in need thereof by contacting a tissue of the subject with one or more transdermal delivery systems. The terms “transdermal” and “topical” are used herein in the broadest sense to refer to administration of an active agent, e.g., memantine or donepezil or fingolimod, to the skin surface or mucosal membrane of an animal, including humans, so that the drug passes through the body surface, e.g., skin, and into the individual's blood stream. The term “transdermal” is intended to include trans-mucosal administration, i.e., administration of a drug to the mucosal (e.g., sublingual, buccal, vaginal, rectal) surface of an individual so that the agent passes through the mucosal tissue and into the individual's blood stream.

The terms “topical delivery system,” “transdermal delivery system” and “TDS,” which refer to the route of delivery of the drug via the skin tissue, are used interchangeably herein.”

The terms “skin” tissue or “cutaneous” tissue as used herein are defined as including tissues covered by a stratum corneum, or stratum lucidum, and/or other mucous membranes. The term further includes mucosal tissue, including the interior surface of body cavities, e.g., buccal, nasal, rectal, vaginal, etc., which have a mucosal lining. The term “skin” should be interpreted as including “mucosal tissue” and vice versa.

Alzheimer's disease is the most common cause of senile dementia and is characterized by cognitive deficits related to degeneration of cholinergic neurons. Alzheimer's affects 6-8% of people over the age of 65 and nearly 30% of people over the age of 85 (Sozio et al., Neurophsychiatric Disease and Treatment, 2012, 8:361-368), involving the loss of cognitive functioning and behavioral abilities. The causes of Alzheimer's disease are not yet fully understood. As Alzheimer's disease is associated with reduced levels of several cerebral neurotransmitters including acetylcholine (Ach), current treatment includes administering cholinesterase inhibitors. Cholinesterase inhibitors reduce the hydrolysis of acetylcholine in the synaptic cleft by inhibiting cholinesterase and/or butyrylcholinesterase, which increases acetylcholine levels resulting in improved neurotransmission (Id.).

The transdermal devices described herein may be designed for long term use and/or continuous administration of the active agent. The FDA has approved daily oral doses of donepezil of 5 mg, 10 mg, and 23 mg. It will be appreciated that the total dose of the active agent per transdermal device will be determined by the size of the device and the loading of the active agent within the adhesive matrix. In an embodiment, the active agent is donepezil in free base form. Lower drug loading of donepezil base may be effective as compared to the salt form (e.g. donepezil hydrochloride). The ability to include lower drug loading to achieve efficacy results in a lower profile for the device (thinner) and/or smaller size, both of which are desirable to reduce discomfort. In some embodiments, the application period for the transdermal device is between about 1-10 days, 1-7 days, 1-5 days, 1-2 days, 3-10 days, 3-7 days, 3-5 days, 5-10 days, and 5-7 days inclusive. In some embodiments, the active agent is released from the adhesive matrix as a continuous and/or sustained release over the application period.

A method for delivering donepezil base transdermally to a subject is provided. In the method a transdermal delivery system is applied to the skin, and upon application of the transdermal delivery system to the skin of a subject, transdermal delivery of the donepezil base occurs, to provide a systemic blood concentration of the agent (or a metabolite) that at steady state is bioequivalent to administration of the therapeutic agent orally. As discussed below, bioequivalency is established by (a) a 90% confidence interval of the relative mean Cₘₐₓ and AUC of the therapeutic agent administered from the transdermal delivery system and via oral delivery are between 0.80 and 1.25 or between 0.70-1.43, or (b) a 90% confidence interval of the geometric mean ratios for AUC and Cₘₐₓ of the therapeutic agent administered from the transdermal delivery system and via oral delivery are between 0.80 and 1.25 or between 0.70-1.43.

Standard PK parameters routinely used to assess the behavior of a dosage form in vivo (in other words when administered to an animal or human subject) include Cₘₐₓ (peak concentration of drug in blood plasma), Tₘₐₓ (the time at which peak drug concentration is achieved) and AUC (the area under the plasma concentration vs time curve). Methods for determining and assessing these parameters are well known in the art. The desirable pharmacokinetic profile of the transdermal delivery systems described herein comprise but are not limited to: (1) a Cₘₐₓ for transdermally delivered form of the donepezil when assayed in the plasma of a mammalian subject following administration, that is bioequivalent to the Cₘₐₓ or an orally delivered or an intravenously delivered form of the drug, administered at the same dosage; and/or (2) an AUC for transdermally delivered form of donepezil when assayed in the plasma of a mammalian subject following administration, that is preferably bioequivalent to the AUC for an orally delivered or an intravenously delivered form of the drug, administered at the same dosage; and/or (3) a Tₘₐₓ for transdermally delivered form of donepezil when assayed in the plasma of a mammalian subject following administration, that is within about 80-125% of the Tₘₐₓ for an orally delivered or an intravenously delivered form of the drug, administered at the same dosage. Preferably the transdermal delivery system exhibits a PK profile having a combination of two or more of the features (1), (2) and (3) in the preceding sentence. Preferably the transdermal delivery system exhibits a PK profile having one or both of the features (1) and (2).

In the field of pharmaceutical development the term “bioequivalence” will be readily understood and appreciated by the person skilled in the art. Various regulatory authorities have strict criteria and tests for assessing whether or not two drug products are bioequivalent. These criteria and tests are commonly used throughout the pharmaceutical industry and the assessment of bioequivalence is recognized as a standard form of activity in drug development programs where the characteristics and performance of one product are being compared to those of another product. Indeed in seeking approval to market certain types of products (e.g. those evaluated under the FDA's “Abbreviated New Drug Application” procedure), it is a requirement that the follow-on product be shown to be bioequivalent to a reference product.

In one embodiment, the method encompasses providing and/or administering a transdermal delivery system comprising donepezil base to a subject in a fasted state is bioequivalent to administration of the agent (in base or salt form) orally or intravenously to a subject also in a fasted state, in particular as defined by Cₘₐₓ and AUC guidelines given by the U.S. Food and Drug Administration and the corresponding European regulatory agency (EMEA). In another embodiment, the method encompasses providing and/or administering a transdermal delivery system comprising donepezil base to a subject in a fasted state is bioequivalent to administration of the agent (in base or salt form) orally or intravenously to a subject also in a non-fasted or fed state. Under U.S. FDA and Europe's EMEA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). Europe's EMEA previously used a different standard, which required a 90% CI for AUC between 0.80 to 1.25 and a 90% CI for Cₘₐₓ between 0.70 to 1.43. Methods for determining Cₘₐₓ and AUC are well known in the art.

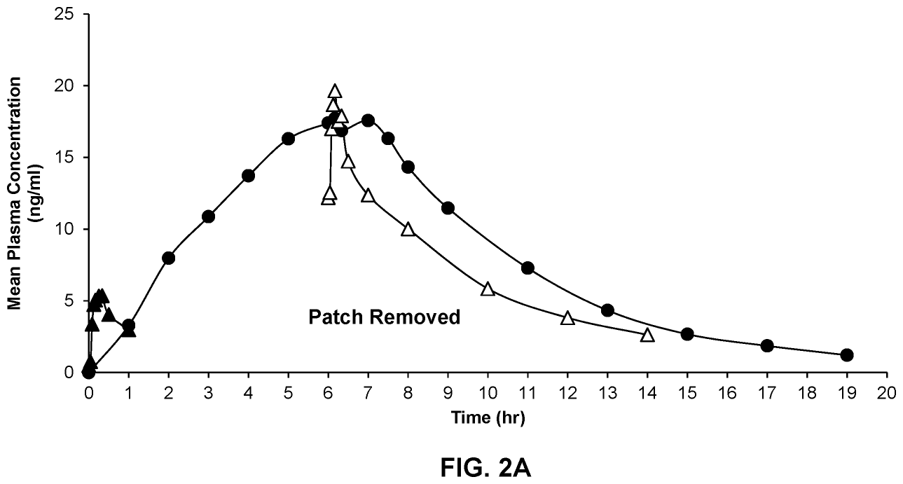

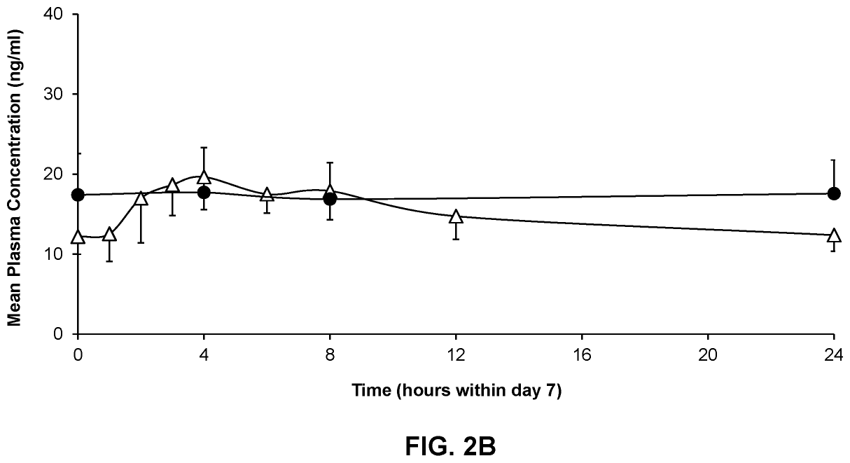

The transdermal delivery system prepared according to Example 1 was tested in vivo for systemic delivery of donepezil, as described in Example 4. In this in vivo study, six human subjects received treatment with a transdermal delivery system applied to their skin and worn for one week, and then removed. Another group of six human subjects were treated with orally administered donepezil (ARICPET®) at a dose of 5 mg taken on day one and on day 7 of the study. Blood samples were taken from the subjects and plasma concentrations of donepezil determined. The results are shown in FIGS. 2A-2B.

FIG. 2A shows the mean plasma concentration of donepezil, in ng/ml, in human subjects treated with a donepezil transdermal delivery system (circles) for 1 week, or with 5 mg of donepezil administered orally on day 1 and on day 7 (triangles). The donepezil transdermal delivery system provided a plasma concentration similar to the plasma concentration provided from oral delivery of a similar dose of donepezil. Accordingly, in one embodiment, a method of administering donepezil transdermally is provided by administering a transdermal delivery system that provides a pharmacokinetic profile that is bioequivalent to the pharmacokinetic profile obtained by oral administration of donepezil.

FIG. 2B is a graph showing a close up of the data points from FIG. 2A in the 24 hour period after oral administration of the 5 mg donepezil tablet (triangles) and after removal of the donepezil transdermal delivery system (circles). The transdermal delivery system provides a sustained, constant donepezil plasma concentration for 24 hours after its removal, similar to that observed in the 24 hour post oral administration.

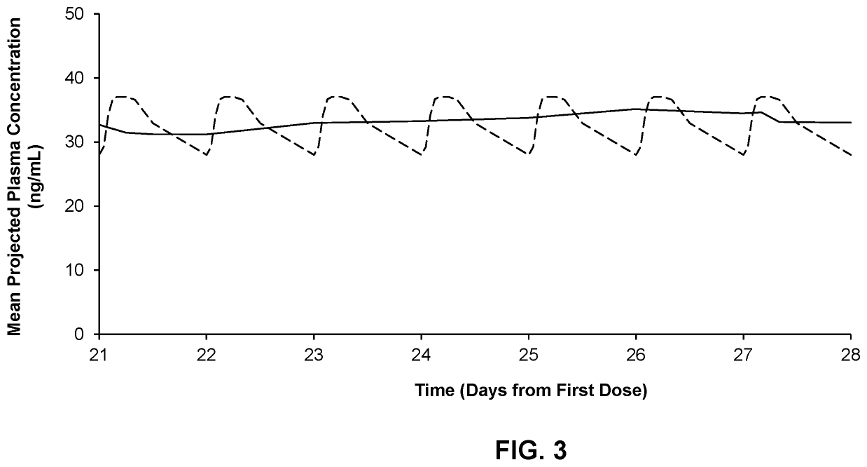

FIG. 3 is a graph showing the projected mean plasma concentration of donepezil, in ng/ml, in the last week of a 28 day (4 week) treatment period with a transdermal delivery system designed to administer 10 mg/day for a week (solid line) and over a 28 day period with a 10 mg daily oral tablet of donepezil (dashed line). The plasma fluctuations resulting from oral administration are eliminated by the transdermal system, where a fresh patch is applied each week and a constant plasma concentration is maintained over the treatment period. The transdermal delivery system provides a constant plasma concentration of donepezil for a sustained period of time (e.g., 3 days, 5 days, 7 days, 14 days), where the plasma concentration is essentially the same as or within about 10%, 15%, 20% or 30% of the plasma concentration achieved with daily oral administration of a similar daily dose of donepezil.

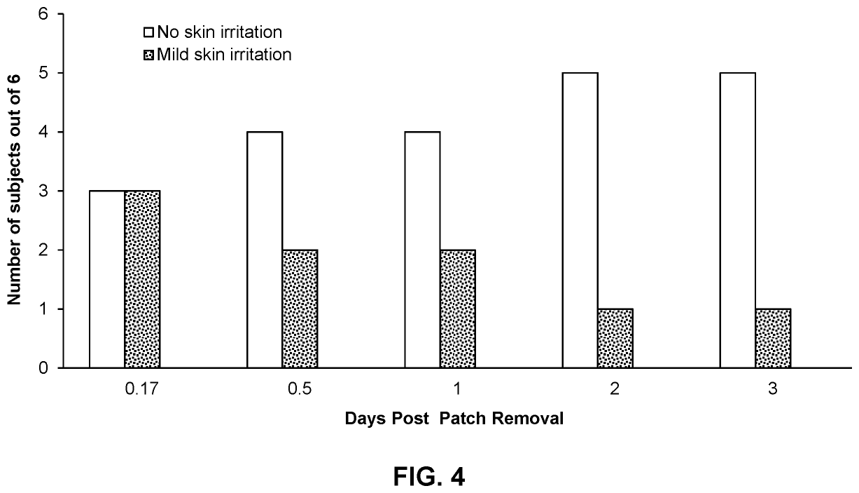

With reference again to the study in Example 4, the six subjects treated with a donepezil transdermal delivery system for one week were monitored for several days following removal of the delivery system from their skin for signs of skin irritation. FIG. 4 is a bar graph showing the number of subjects out of the 6 in the group and the observed skin irritation in the period after removal of the delivery system, where the open bars indicate no skin irritation and the filled bars indicate mild skin irritation. The delivery system resulted in no or mild skin irritation in the hours after removal, and any mild irritation resolved with a day or two.

In another study, human subjects were treated with a transdermal delivery system designed to deliver systemically an amount of donepezil that is bioequivalent to orally administered donepezil at a 10 mg, once daily dose. The projected pharmacokinetic parameters Cₘₐₓ, AUC and Cₘᵢₙ for the two routes of delivery are compared in Table 1.

TABLE 1
Projected Pharmacokinetic Parameters

| | Once-weekly | | |
|---|---|---|---|
| | transdermal | 10 mg oral | Geometric |
| PK Parameter | delivery | donepezil, | Mean Ratio |
| at Steady State | system | once daily | (transdermal:oral) |
| Geometric mean Cₘₐₓ | 40.6 | 45.6 | 0.890 |
| (ng/ml) | | | |
| Geometric mean Cₘᵢₙ | 34.2 | 30.8 | 1.110 |
| (ng/ml) | | | |
| Geometric mean | 6367 | 6165* | 1.033 |
| AUC_{week} (ng-hr/ml) | | | |

Accordingly, in one embodiment, a method for delivering donepezil base to a subject is provided. The method comprises providing a transdermal delivery system comprised of donepezil, and administering or instructing to administer the transdermal delivery system to the skin of a subject. The method achieves transdermal delivery of donepezil that is bioequivalent to administration of the therapeutic agent orally, wherein bioequivalency is established by (a) a 90% confidence interval of the relative mean Cₘₐₓ and AUC of the therapeutic agent administered from the transdermal delivery system and via oral delivery between 0.70-1.43 or between 0.80 and 1.25, or (b) a 90% confidence interval of the ratios for AUC and Cₘₐₓ of the therapeutic agent administered from the transdermal delivery system and via oral delivery between 0.70-1.43 or between 0.80 and 1.25.

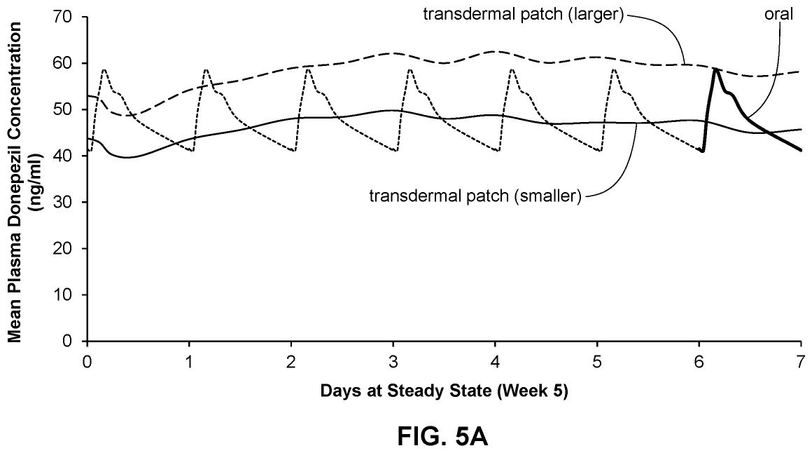

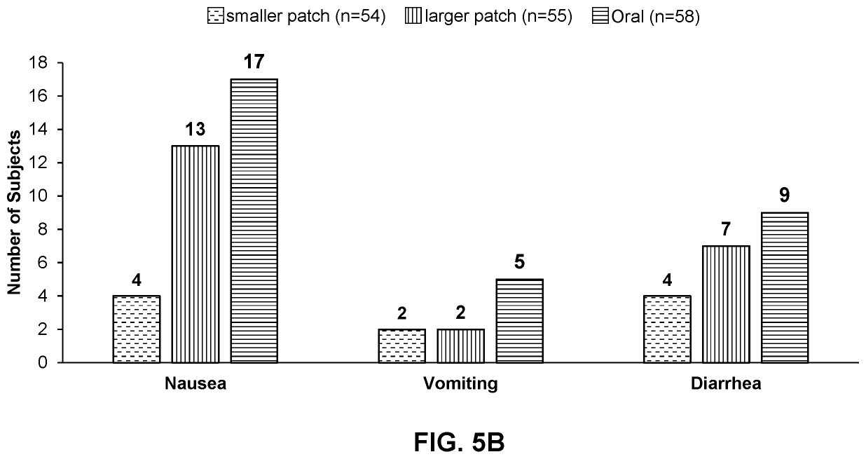

Example 5 describes a study conducted on human subjects where transdermal patches comprising donepezil were studied and compared to orally administered donepezil. In this study, patients were enrolled to participate in a six month, three-period, randomized crossover study comparing the steady-state pharmacokinetic profiles of once-daily oral donepezil (ARICEPT®) with a donepezil transdermal patch formulation. The transdermal patch was provided in two sizes, A and B, yet other than surface area, the transdermal patches were the same in all respects. During the study, the participants in each treatment arm received one week of 5 mg/day of donepezil, followed by 4 weeks of 10 mg/day of donepezil delivered from a once-weekly transdermal patch of two sizes (Arm 1 and Arm 2) or orally (Arm 3). Pharmacokinetic measurements were evaluated during the fourth week of the 10 mg/day treatment, when plasma concentrations had achieved steady levels. Blood samples for the subjects receiving the transdermal treatment were taken daily throughout the fourth week to determine pharmacokinetics. Subjects receiving oral donepezil had blood drawn on the last day of the fourth week to determine pharmacokinetics. The mean plasma concentration of donepezil, in ng/mL, is shown in FIG. 5A, for each day in week 5 of the study, where the solid line corresponds to the transdermal patch with a smaller surface area and the dashed line corresponds to the transdermal patch with a larger surface area. The thick, bold line at days 6-7 shows the mean plasma concentration for the subjects receiving the oral donepezil, and the dotted line shows the projected daily plasma concentration for oral treatment. The mean plasma concentrations of donepezil in the subjects treated with a transdermal patch were bioequivalent to the plasma concentration of donepezil in the subject treated orally with donepezil. The larger and smaller transdermal patches demonstrated dose proportionality. Table 2 shows the primary pharmacokinetic parameters in a bioequivalence assessment of the smaller surface area transdermal patch used in the study.

TABLE 2

| | Geometric | |
|---|---|---|
| Primary | Mean Ratio (%) | Bioequivalence |
| Pharmacokinetic | of smaller patch | Limits |
| Parameter | to oral dose | (target 80-125%) |
| AUC (ng-hr/mL) | 104.7% | 95.2-115.2 |
| Cmaxₛₛ (ng/mL) | 91.6% | 83.1-100.8 |

The gastrointestinal related adverse events of nausea, vomiting and diarrhea reported by the subjects in the clinical study mentioned above with respect to FIG. 5A are shown in FIG. 5B. Subjects treated with the smaller size transdermal patch (bars with dashed fill) and with the larger size transdermal patch (bars with vertical line fill) had a lower incidence or nausea, vomiting and diarrhea than subjects treated with oral (bars with horizontal line fill) donepezil. The number of subjects experiencing nausea was four-fold lower when the 10 mg/day donepezil was administered transdermally versus orally. The number of subjects experiencing diarrhea was two-fold lower when 10 mg/day donepezil was administered transdermally versus orally.

Accordingly, in one embodiment, a composition and a method for delivering donepezil to a subject is provided. The composition, when applied to the skin of a subject, provides transdermal delivery of donepezil to achieve a plasma concentration of donepezil that at steady state is bioequivalent to administration of donepezil orally, and/or that provides a number of gastrointestinal related adverse events that is two-fold, three-fold, four-fold, or five-fold lower than subjects treated with the same dose of donepezil orally (i.e., the dose given orally is equal to the dose given transdermally, such that the subjects are treated with an equal dose of donepezil given orally or transdermally). In one embodiment, the donepezil given orally is a salt form of donepezil and the donepezil given transdermally is donepezil base. In one embodiment, the number of gastrointestinal related adverse events is between 2-5, 2-4, and 2-3 fold lover, and in another embodiment, the number of gastrointestinal related adverse events is at least about two-fold, at least about three-fold, at least about four-fold, or at least about five-fold lower than subjects treated with the same dose of donepezil orally. In one embodiment, the delivery of donepezil is for the treatment of Alzheimer's disease.

The transdermal devices described herein may be designed for long term use and/or continuous administration of the active agent. The FDA has approved doses of memantine of 2 mg, 5 mg, 7 mg, 10 mg, 14 mg, 21 mg, and 28 mg. It will be appreciated that the total dose of the active agent per transdermal device will be determined by the size of the device and the loading of the active agent within the adhesive matrix. In an embodiment, the active agent is memantine in free base form. Lower drug loading of memantine may be effective as compared to the salt form (e.g. memantine hydrochloride). The ability to include lower drug loading to achieve efficacy results in a lower profile for the device (thinner) and/or smaller size, both of which are desirable to reduce discomfort. In some embodiments, the application period for the transdermal device is between about 1-10 days, 1-7 days, 1-5 days, 1-2 days, 3-10 days, 3-7 days, 3-5 days, 5-10 days, and 5-7 days inclusive. In some embodiments, the active agent is released from the adhesive matrix as a continuous and/or sustained release over the application period.

A method for delivering memantine transdermally to a subject is provided. In the method a transdermal delivery system is applied to the skin, and upon application of the transdermal delivery system to the skin of a subject, transdermal delivery of the memantine occurs, to provide a systemic blood concentration of the agent (or a metabolite) that at steady state is bioequivalent to administration of the therapeutic agent orally. As discussed below, bioequivalency is established by (a) a 90% confidence interval of the relative mean Cₘₐₓ and AUC of the therapeutic agent administered from the transdermal delivery system and via oral delivery are between 0.80 and 1.25, or (b) a 90% confidence interval of the ratios for AUC and Cₘₐₓ of the therapeutic agent administered from the transdermal delivery system and via oral delivery are between 0.80 and 1.25.

Standard pharmacokinetic (PK) parameters routinely used to assess the behavior of a dosage form in vivo (in other words when administered to an animal or human subject) include Cₘₐₓ (peak concentration of drug in blood plasma), Tₘₐₓ (the time at which peak drug concentration is achieved) and AUC (the area under the plasma concentration vs time curve). Methods for determining and assessing these parameters are well known in the art. The desirable pharmacokinetic profile of the transdermal delivery systems described herein comprise but are not limited to: (1) a Cₘₐₓ for transdermally delivered form of the memantine when assayed in the plasma of a mammalian subject following administration, that is bioequivalent to the Cₘₐₓ or an orally delivered or an intravenously delivered form of the drug, administered at the same dosage; and/or (2) an AUC for transdermally delivered form of memantine when assayed in the plasma of a mammalian subject following administration, that is preferably bioequivalent to the AUC for an orally delivered or an intravenously delivered form of the drug, administered at the same dosage; and/or (3) a Tₘₐₓ for transdermally delivered form of memantine when assayed in the plasma of a mammalian subject following administration, that is within about 80-125% of the Tₘₐₓ for an orally delivered or an intravenously delivered form of the drug, administered at the same dosage. Preferably the transdermal delivery system exhibits a PK profile having a combination of two or more of the features (1), (2) and/or (3) in the preceding sentence. In another embodiment, the transdermal delivery system exhibits a PK profile having a combination of one or both of the features (1) and (2).

In the field of pharmaceutical development the term “bioequivalence” will be readily understood and appreciated by the person skilled in the art. Various regulatory authorities have strict criteria and tests for assessing whether or not two drug products are bioequivalent. These criteria and tests are commonly used throughout the pharmaceutical industry and the assessment of bioequivalence is recognized as a standard form of activity in drug development programs where the characteristics and performance of one product are being compared to those of another product. Indeed in seeking approval to market certain types of products (e.g. those evaluated under the FDA's “Abbreviated New Drug Application” procedure), it is a requirement that the follow-on product be shown to be bioequivalent to a reference product.

In one embodiment, the method encompasses providing and/or administering a transdermal delivery system comprising memantine base to a subject in a fasted state is bioequivalent to administration of the agent (in base or salt form) orally or intravenously to a subject also in a fasted state, in particular as defined by Cₘₐₓ and AUC guidelines given by the U.S. Food and Drug Administration and the corresponding European regulatory agency (EMEA). Under U.S. FDA and

Europe's EMEA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). Europe's EMEA previously used a different standard, which required a 90% CI for AUC between 0.80 to 1.25 and a 90% CI for Cₘₐₓ between 0.70 to 1.43. Methods for determining Cₘₐₓ and AUC are well known in the art.

Accordingly, in one embodiment, a method for delivering memantine base to a subject is provided. The method comprises providing a transdermal delivery system comprised of memantine, and administering or instructing to administer the transdermal delivery system to the skin of a subject. The method achieves transdermal delivery of memantine that is bioequivalent to administration of the therapeutic agent orally, wherein bioequivalency is established by (a) a 90% confidence interval of the relative mean Cₘₐₓ and AUC of the therapeutic agent administered from the transdermal delivery system and via oral delivery between 0.70 and 1.43 or between 0.80 and 1.25, or (b) a 90% confidence interval of the geometric mean ratios for AUC and Cₘₐₓ of the therapeutic agent administered from the transdermal delivery system and via oral delivery between 0.70 and 1.43 or between 0.80 and 1.25.

Examples 6 and 7 set forth further exemplary compositions and delivery systems. As described in Example 6, a transdermal delivery system is prepared comprising a drug reservoir layer and a contact adhesive layer with a rate controlling membrane layer situated between the drug reservoir and the contact adhesive layers, as depicted in FIG. 1A. A drug reservoir in the form of a solid monolithic adhesive reservoir is prepared using an acrylic acid/vinyl acetate copolymer adhesive and cross-linked polyvinylpyrrolidone (PVP-CLM), along with the named dissolving agents, carriers and optionally permeation enhancers (Table 3). The drug reservoir contains approximately 25 wt % memantine hydrochloride and 9.73 wt % sodium bicarbonate, to generate in situ memantine base. A contact adhesive layer containing higher alcohol and biocompatible polymer is synthesized. In a second variant, the contact adhesive contained the higher alcohol and biocompatible polymer, along with dispersive silica. To control the diffusional release of memantine base from the drug reservoir, a rate-controlling membrane may be introduced in between the drug reservoir and the contact adhesive.

TABLE 3
Transdermal delivery systems, with
two contact adhesive formulations

| | | Contact | Contact |
|---|---|---|---|
| | Drug | Adhesive | Adhesive |
| | Reservoir | #1 | #2 |
| | Dry | Dry | Dry |
| | Composition | Composition | Composition |
| COMPONENTS | (%) | (%) | (%) |
| Memantine HCl | 25% | 0 | 0 |
| Sodium bicarbonate | 9.73% | 0 | 0 |
| Octyldodecanol | 10% | 10% | 10% |
| Glycerol | 10% | 0 | 0 |
| fumed silica | 0 | 0 | 7% |
| (AEROSIL ® 200) | | | |
| crosslinked | 15% | 20% | 0 |
| polyvinylpyrrolidone | | | |
| (KOLLIDON ® CL-M) | | | |
| acrylic acid/vinyl | 30.3% | 0 | 0 |
| acetate copolymer | | | |
| (DURO-TAK ® | | | |
| 387/87-2287) | | | |
| Polyisobutylene/ | 0 | 70 | 83% |
| polybutene | | | |
| Total | 100% | 100% | 100% |

As described in Example 6, transdermal delivery systems are prepared and are comprised of a drug reservoir and a skin contact adhesive layer separated by an intermediate layer. The drug reservoir in the exemplary systems comprises the copolymer acrylic acid/vinyl acetate and cross-linked polyvinylpyrrolidone (KOLLIDON-CLM). These base materials are mixed with the named carriers and dissolving agents, memantine hydrochloride and sodium bicarbonate (Table 4). The drug reservoir contains approximately 25 wt % memantine hydrochloride and 9.73 wt % sodium bicarbonate, to generate in situ memantine base. The skin contact adhesive layer contains a higher alcohol and biocompatible polymer.

TABLE 4
Transdermal delivery system

| | Drug | Contact |
|---|---|---|
| | Reservoir | Adhesive |
| | Dry | Dry |
| | Composition | Composition |
| | (%) | (%) |
| Memantine HCl | 25% | 0 |
| Sodium bicarbonate | 9.7% | 0 |
| Octyldodecanol | 7% | 10% |
| Glycerol | 10% | 0 |
| crosslinked | 15% | 20% |
| polyvinylpyrrolidone | | |
| (KOLLIDON ® CL-M) | | |
| acrylic acid/vinyl acetate | 33.3% | 0 |
| copolymer (DURO-TAK ® | | |
| 387/87-2287) | | |
| polyisobutylene/polybutene | 0 | 70% |
| Total | 100% | 100% |

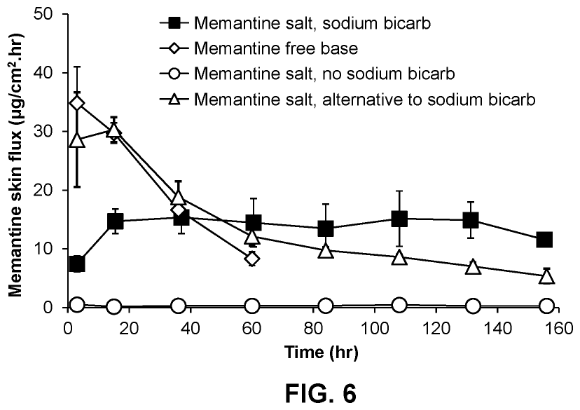

A memantine transdermal system was prepared as described in Example 7 to demonstrate the delivery of an active agent formulated from an amine salt form of the active agent and an amphoteric inorganic base compound. The memantine transdermal system was evaluated in vitro by measuring release of memantine from the system and across human skin and the results are shown in FIG. 6 (squares). About 18 hours after application of the transdermal system to the skin, a steady-state flux rate of between about 12-15 μg/cm²-hr was achieved. The flux rate remained steady for about 6.5 days before decreasing. Accordingly, in one embodiment, a transdermal delivery system for delivery of a base form of an active agent is prepared from an amine salt form of the active agent and sodium bicarbonate, to provide a skin flux rate or permeation rate that is therapeutic for a period of at least about 3 days or 5 days or 7 days (or from 3-7 days). In one embodiment, the steady state in vitro skin flux rate remains within 15%, 20%, 25%, or 30% for a period of at least about 3 days or 5 days or 7 days (or from 3-7 days). That is, the in vitro skin flux measured at time point y varies from an in vitro skin flux measured at an earlier adjacent time point x, where x and y are each time points within a 3 day, 5 day, or 7 day measurement period, by less than 15%, 20%, 25% or 30%.

Comparative examples were also conducted to illustrate the inventive composition, system and methods described herein. FIG. 6 illustrates that adhesive compositions (transdermal systems) prepared with the free base form of the drug (diamond), with the amine salt form of drug but without sodium bicarbonate (circle) or a salt form of an amine drug and an amphoteric inorganic base compound, but where the pKa of the amphoteric inorganic base compound is not lower than that of the amine salt form of the active agent but is higher (triangle). In these comparative examples, the in vitro skin flux of the drug is insufficient for therapy.

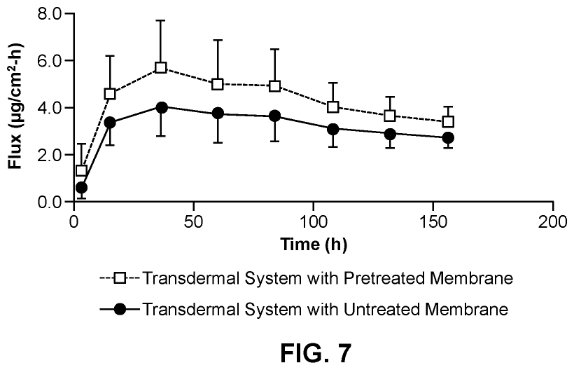

A transdermal system for delivery of donepezil comprising a microporous membrane layer that has been pretreated with a membrane treatment composition is described in Example 9. A comparative example of a transdermal system in which the microporous membrane was left untreated is also described. Comparative in vitro skin flux studies were performed and the results are provided in FIG. 7. It can be seen that the treatment of the microporous membrane with a membrane treatment composition increases the total skin flux of donepezil and that this flux is maintained over an extended period of time.

### IV. Examples

The following examples are illustrative in nature and are in no way intended to be limiting.

### Example 1

### Donepezil Transdermal Delivery System

A transdermal delivery system comprising donepezil was prepared as follows.

### Preparation of Drug Reservoir

Sorbitan monolaurate (SPAN® 20, 1.20 grams) was dissolved in 6.00 g of triethyl citrate and mixed with 1.80 grams of lauryl lactate and 89.69 grams of ethyl acetate. 6.00 grams of glycerin was added and mixed. 9.00 grams of donepezil hydrochloride and 1.82 grams of sodium bicarbonate were added and dispersed in the mixture. 12.00 grams of crosslinked, micronized polyvinylpyrrolidone (Kollidon® CL-M) was then added and the mixture was homogenized. To the homogenized drug dispersion, 43.93 grams of acrylic acid/vinyl acetate copolymer (Duro-Tak® 387-2287, solid content 50.5%) was added and well mixed. The wet adhesive formulation was coated on a release liner and dried using a lab coater (Werner Mathis) to yield a dry coat weight of 12 mg/cm².

### Preparation of Contact Adhesive:

Sorbitan monolaurate (SPAN® 20, 0.60 grams) was dissolved in 3.0 grams of triethyl citrate and mixed with 0.9 grams of lauryl lactate, 25.45 grams of ethyl acetate and 1.34 grams of isopropyl alcohol. 6.00 grams of crosslinked, micronized polyvinylpyrrolidone (Kollidon® CL-M) was added and the mixture was homogenized. To the homogenized mixture 38.61 grams of acrylic acid/vinyl acetate copolymer (Duro-Tak® 387-2287, solid content 50.5%) was added and mixed well. The wet adhesive formulation was coated on a release liner and dried to give a dry coat weight of 5 mg/cm².

### Lamination and Die-Cut

A rate controlling membrane (CELGARD® 2400 or Reemay® 2250) was laminated on the adhesive side of the drug reservoir. Then the contact adhesive was laminated on top of the A rate controlling membrane laminated with drug reservoir. The release liner on the drug reservoir side was replaced and laminated with backing film. The final five layer laminate was die-cut into transdermal patches.

The weight percentage of the components in the transdermal delivery system are set forth in Table 1.1 below.

TABLE 1.1

| | | | total |
|---|---|---|---|
| | wt. % | wt. % | wt. % in |
| | in drug | in contact | delivery |
| Ingredient | reservoir | adhesive | system |
| Donepezil HCl | 5.2% | — | 3.6% |
| Sodium bicarbonate | 1.1% | — | 0.74% |
| sorbitan monolaurate | 0.7% | 0.8% | 0.73% |
| (Span ® 20) | | | |
| Triethyl citrate | 3.5% | 3.9% | 3.6% |
| Lauryl lactate | 1.05% | 1.2% | 1.1% |
| Ethyl acetate | 52.3% | 33.5% | 46.6% |
| Glycerin | 3.5% | — | 2.4% |
| crosslinked, micronized | 7.0% | 7.9% | 7.3% |
| polyvinylpyrrolidone | | | |
| (Kollidon ® CL-M) | | | |
| acrylic acid/vinyl | 25.6% | 50.9% | 33.4% |
| acetate copolymer | | | |
| (Duro-Tak ® 387-2287) | | | |
| isopropyl alcohol | — | 1.8% | 0.54% |

### Example 2

### Donepezil Transdermal Delivery Systems

Transdermal delivery system comprising donepezil was prepared as follows.

### Preparation of Drug Reservoir

Sorbitan monolaurate (SPAN® 20) was dissolved in triethyl citrate and mixed with lauryl lactate. Glycerin was added and mixed. Donepezil hydrochloride and sodium bicarbonate were added and dispersed in the mixture. Crosslinked, micronized polyvinylpyrrolidone (KOLLIDON® CL-M) was then added and the mixture was homogenized. To the homogenized drug dispersion, acrylic acid/vinyl acetate copolymer (DURO-TAK® 387-2287, solid content 50.5%) was added and well mixed. The wet adhesive formulation was coated on a release liner and dried using a lab coater (Werner Mathis).

### Preparation of Contact Adhesive

Sorbitan monolaurate (SPAN® 20) was dissolved in triethyl citrate and mixed with lauryl lactate. Crosslinked, micronized polyvinylpyrrolidone (Kollidon® CL-M) was added and the mixture was homogenized. To the homogenized mixture acrylic acid/vinyl acetate copolymer (DURO-TAK® 387-2287, solid content 50.5%) was added and mixed well. The wet adhesive formulation was coated on a release liner and dried.

### Lamination and Die-Cut

A rate controlling membrane (CELGARD® 2400) was laminated on the adhesive side of the drug reservoir. Then the contact adhesive was laminated on top of the rate controlling membrane laminated with drug reservoir. The release liner on the drug reservoir side was replaced and laminated with backing film. The final five layer laminate was die-cut into transdermal patches.

The weight percentage of the components in the transdermal delivery systems are set forth in Table 2.1 below.

TABLE 2.1

| | Drug | Contact |
|---|---|---|
| | Reservoir | Adhesive |
| | (Dry Formula | (Dry formula, |
| Ingredient | % wt/wt) | % wt/wt) |
| Donepezil HCl | 16.0 | 0 |
| Sodium bicarbonate | 2.6 | 0 |
| Triethyl citrate | 10.0 | 10.0 |
| Lauryl Lactate | 3.0 | 3.0 |
| Sorbitan monolaurate | 2.0 | 2.0 |
| (SPAN ® 20) | | |
| Glyerine | 10.0 | 0 |
| PVP-CLM | 15.0 | 20.0 |
| (KOLLIDONE ®-CLM) | | |
| acrylic acid/vinyl acetate | 41.4 | 65.0 |
| copolymer (Duro-Tak ® | | |
| 387-2287) | | |

### Example 3

### Donepezil Transdermal Delivery Systems

Transdermal delivery system comprising donepezil was prepared as follows.

### Preparation of Drug Reservoir:

Sorbitan monolaurate (SPAN® 20) was dissolved in triethyl citrate and mixed with lauryl lactate. Glycerin was added and mixed. Donepezil hydrochloride was added and dispersed in the mixture. Fumed silica (AEROSIL® 200 Pharma) was then added and the mixture was homogenized. To the homogenized drug dispersion, acrylic acid/vinyl acetate copolymer (DURO-TAK® 387-2287, solid content 50.5%) and dimethylaminoethyl methacrylate, butyl methacrylate, methyl methacrylate copolymer (EUDRAGIT® EPO) were added and well mixed. The wet adhesive formulation was coated on a release liner and dried using a lab coater (Werner Mathis).

### Preparation of Contact Adhesive:

Sorbitan monolaurate (SPAN® 20) was dissolved in triethyl citrate and mixed with lauryl lactate. Crosslinked, micronized polyvinylpyrrolidone (KOLLIDON® CL-M) was added and the mixture was homogenized. To the homogenized mixture acrylic acid/vinyl acetate copolymer (Duro-Tak® 387-2287, solid content 50.5%) added and mixed well. The wet adhesive formulation was coated on a release liner and dried.

### Lamination and Die-Cut

A rate controlling membrane (CELGARD® 2400) was laminated on the adhesive side of the drug reservoir. Then the contact adhesive was laminated on top of the rate controlling membrane laminated with drug reservoir. The release liner on the drug reservoir side was replaced and laminated with backing film. The final five layer laminate was die-cut into transdermal patches.

The weight percentage of the components in the transdermal delivery systems are set forth in Table 3.1 below.

TABLE 3.1

| | Drug | Contact |
|---|---|---|
| | Reservoir | Adhesive |
| | (Dry Formula | (Dry formula, |
| Ingredient | % wt/wt) | % wt/wt) |
| Donepezil HCl | 25.0 | 0 |
| dimethylaminoethyl | 17.7 | 0 |
| methacrylate, butyl | | |
| methacrylate, methyl | | |
| methacrylate copolymer | | |
| (EUDRAGIT ® EPO) | | |
| Triethyl citrate | 10.0 | 10.0 |
| Lauryl Lactate | 6.0 | 6.0 |
| Sorbitan monolaurate | 2.0 | 2.0 |
| (SPAN ® 20) | | |
| fumed silica | 7.0 | 0 |
| (AEROSIL ® 200 Pharma) | | |
| Glyerine | 10.0 | 0 |
| PVP-CLM | 0 | 20.0 |
| (KOLLIDONE ®-CLM) | | |
| acrylic acid/vinyl acetate | 24.3 | 64.0 |
| copolymer | | |
| (Duro-Tak ® 387-2287) | | |

### Example 4

In Vivo Administration of Donepezil from a Donepezil Transdermal Delivery System

Transdermal delivery systems comprising donepezil were prepared as described in Example 1. Twelve (12) human subjects were randomized into two groups for treatment with a transdermal delivery system (n=6) or with orally administered donepezil (ARICPET®), 5 mg taken on day one and on day 7 of the study. The transdermal delivery system was applied to the skin and worn for one week and then removed. Blood samples were taken daily from the subjects treated with the transdermal delivery system. Blood samples were taken at frequent hour intervals on day 1 and day 7 in the group treated with orally delivered donepezil, and again on days 8, 10, 12 and 14. Mean plasma concentration of donepezil in the treatment groups are shown in FIGS. 2A-2B.

### Example 5

In Vivo Administration of Donepezil from a Donepezil Transdermal Delivery System

Transdermal delivery systems comprising donepezil were prepared as described in Example 2. Patients were enrolled and randomly separated into three treatment arms for a five week treatment study. The patients in Arm 1 (n=52) and Arm 2 (n=51) were treated with a transdermal system of Example 2, where the patients in Arm 1 wore a patch having a smaller surface area (Patch A) than the patients in Arm 2 (Patch B). Other than size, Patch A and Patch B were identical. In the first week of the study, patients in Arm 1 and Arm 2 wore patches designed to deliver 5 mg donepezil from a once-weekly patch. After the initial 7 day period, the patients were given a transdermal system designed to be worn for 7 days (once-weekly transdermal patch) to deliver 10 mg donepezil per day, again with Patch A differing from Patch B only in surface area. The transdermal systems were replaced weekly for 4 weeks. The patients in Arm 3 (n=54) were treated with a daily oral dose of 5 mg donepezil (ARICEPT) for 7 days followed by a once daily 10 mg dose of donepezil (ARICEPT) for 4 weeks.

For the subjects in Arm 1 and Arm 2, blood samples were taken daily during the fourth week of dosing at the 10 mg level, when plasma concentrations were at steady state. For the subjects in Arm 3, blood samples were taken on the last day of the fourth week of 10 mg/day dosing. The mean plasma concentration of donepezil for the treatment arms in the fourth week of the 10 mg dosing are shown in FIG. 5A, where subjects treated with donepezil administered transdermally from transdermal Patch A (smaller surface area, solid line, transdermal Patch B (larger surface area, dashed line) and oral donepezil (thick, bold line at days 6-7) are shown, along with a dotted line showing the projected daily plasma concentration for oral treatment.

FIG. 5B is a bar graph showing the number of gastrointestinal related adverse events (nausea, vomiting and diarrhea) reported by subjects in the study, where bars with dashed fill correspond to subjects treated with the weekly smaller size transdermal patch, the bars with vertical line fill correspond to subjects treated with the weekly larger size transdermal patch, and the bars with horizontal line fill correspond to the subjects treated with oral donepezil.

### Example 6

### Memantine Transdermal Delivery System

A transdermal delivery system comprising memantine is prepared as follows.

### Preparation of Drug Reservoir:

A memantine salt and an alkaline salt are dissolved in a mixture of ethyl acetate, about isopropyl alcohol, propylene glycol, and levulinic acid, to form a clear solution. In one variation, fumed silica (AEROSIL® 200P) is added and the mixture is homogenized. To the homogenous mixture, a copolymer of acrylic acid/vinyl acetate (DURO-TAK® 387-2287) is added and mixed until the mixture becomes homogenous.

The adhesive formulation mixture is coated on a siliconized polyethylene terephthalate liner and dried in a Werner Mathis coater at 60° C. for 8 minutes to yield a dry adhesive layer.

A transdermal delivery system is fabricated using two of the dry adhesive layers sandwiched together with a non-woven polyester fabric between the two adhesive layers. Then, coated polyethylene terephthalate liner is replaced with a backing film.

### Preparation of Contact Adhesive

Octyldodecanol, crosslinked, micronized polyvinylpyrrolidone (KOLLIDON® CL-M), and an optional solvent are mixed and the mixture is homogenized. To the homogenized mixture, polyisobutylene (PIB, 10/50/40) is added and mixed well. The wet adhesive formulation is coated on a release liner and dried.

### Lamination and Die-Cut

An intermediate layer (CELGARD® 2400 or Reemay® 2250) is laminated on the adhesive side of the drug reservoir. Then the contact adhesive is laminated on top of the rate controlling membrane laminated with the drug reservoir. The release liner on the drug reservoir side is replaced and laminated with a backing film.

Transdermal delivery systems are then die-cut from the laminate.

### Example 7

Memantine Salt Transdermal Formulation with Sodium Bicarbonate

### Preparation of Drug-in-Adhesive (Drug Reservoir)

An amount of 2.0 g of glycerine and 2.0 g of octyl dodecanol were mixed with a mixture of 29.35 g of ethyl acetate and 1.86 g of isopropyl alcohol. In the solution, 5.0 g of memantine hydrochloride and 1.95 g of sodium bicarbonate were dispersed by stirring. To the dispersion, 3.0 g of crosslinked polyvinylpyrrolidone (KOLLIDON® CL-M) was added and homogenized using a Silverson mixer homogenizer. To the homogenized dispersion, 11.99 g of acrylate copolymer (DURO-TAK® 387-2287, solid content 50.5%) was added and mixed well. The wet adhesive formulation was coated on a release liner and dried using a Werner Mathis coater to get a dry coat weight of 15 mg/cm².

### Preparation of Contact Adhesive

An amount of 2.0 g of octyl dodecanol was mixed with 20.67 g of n-heptane. After addition of 4.00 g of crosslinked polyvinylpyrrolidone (KOLLIDON® CL-M) to the solution, the mixture was homogenized using a Silverson mixer homogenizer. To the homogenized mixture, an amount of 23.33 g of polyisobutylene adhesive solution (solid content 60%) was added and mixed well. The wet adhesive formulation was coated on a release liner and dried to give a dry coat weight of 5 mg/cm².

### Lamination and Die-Cut

A polypropylene microporous membrane (Celgard® 2400) was laminated between the drug-in-adhesive layer and the contact adhesive layer. The release liner on the drug-in-adhesive side was replaced and laminated with a backing, 3M SCOTCHPAK® 1012. The final five layer laminate was die-cut into patches.

### Evaluation of In Vitro Skin Flux

Dermatomed human cadaver skin was obtained from a skin bank and frozen until ready for use. The skin was placed in water at 60° C. for 1-2 mins minute after thawing and the epidermis carefully separated from dermis. The epidermis was either used immediately or wrapped and frozen for later use.

In vitro skin flux studies were performed using a Franz type diffusion cell with an active diffusion area of 0.64 cm². The epidermis was mounted between the donor and receptor compartments of the diffusion cell. The transdermal delivery system was placed over the skin and the two compartments were clamped tight together.

The receptor compartment was filled with 0.01 M phosphate buffer, pH 6.5, containing 0.01% gentamicin. The solution in the receptor compartment was continually stirred using a magnetic stirring bar in the receptor compartment. The temperature was maintained at 32±0.5° C. Samples were drawn from the receptor solution at periodic intervals and the receptor solution was replaced with fresh phosphate buffers solution. Drug content in the samples was analyzed using LCMS for memantine.

The flux profile results are shown in FIG. 7 (squares). The flux in this example is relatively high and remains relatively constant over 7 days.

### Example 8

In Vivo Administration of Memantine with Transdermal Delivery System

Transdermal delivery systems comprising memantine are prepared as described in Example 1. Human subjects are randomized into two groups for treatment with a transdermal delivery system or with orally administered memantine (NAMENDA®), 7 mg taken on day one and on day 7 of the study. The transdermal delivery system is applied to the skin and worn for one week and then removed. Blood samples are taken daily from the subjects treated with the transdermal delivery system. Blood samples were taken at frequent hour intervals on day 1 and day 7 in the group treated with orally delivered memantine, and again on days 8, 10, 12 and 14. Mean plasma concentration of memantine in the treatment groups are measured.

### Example 9

Donepezil HCl Transdermal System with Microporous Membrane
Pretreatment of Microporous Membrane with a Membrane Treatment Composition

A polypropylene microporous membrane (Celgard® 2400) having a typical porosity 41% and pore size 0.043 μm was used as the microporous membrane in this example. Two different donepezil patches were prepared, one with pre-treated polypropylene microporous membrane and the other with untreated membrane to compare the in vitro skin flux profiles of the two systems.

A membrane treatment composition of 66.67% w/w of triethyl citrate, 20.00% w/w of lauryl lactate, and 13.33% w/w of sorbitan monolaurate was prepared. The triethyl acetate was mixed well with lauryl lactate to form a clear solution. The sorbitan monolaurate was then added to the mixture and mixed well by a high shear stirring to form a cloudy homogeneous composition. The cloudy liquid was then coated on the membrane with a coating knife to saturate it with the liquid mixture. When saturated, the initially white membrane turned into a translucent membrane. Excess membrane treatment composition was then removed by wiping away.

### Preparation of Drug Reservoir

An amount of 1.20 grams of sorbitan monolaurate (SPAN® 20) was dissolved in a mixture of 6.00 g of triethyl citrate, and mixed with 1.80 grams of lauryl lactate and 89.69 grams of ethyl acetate. 6.00 grams of glycerin was added and mixed. To the mixture, 9.00 grams of donepezil hydrochloride and 1.82 grams of sodium bicarbonate were dispersed. After addition of 12.00 grams of cross linked polyvinylpyrrolidone (Kollidon® CL-M) to the drug dispersed solution, the mixture was homogenized well. To the homogenized drug dispersion, 43.93 grams of acrylate copolymer (Duro-Tak® 387-2287, solid content 50.5%) was added and well mixed. Ascorbic palmitate was added. The wet adhesive formulation was coated on a release liner and dried using a lab coater (Werner Mathis coater) to get a dry coat weight of 12 mg/cm².

### Preparation of Contact Adhesive

An amount of 0.60 grams of sorbitan monolaurate (SPAN® 20) was dissolved in 3.00 grams of triethyl citrate, and mixed with 0.9 grams of lauryl lactate, 25.45 grams of ethyl acetate, and 1.34 grams of isopropyl alcohol. After addition of 6.00 grams of cross linked polyvinylpyrrolidone (Kollidon® CL-M) the mixture was homogenized. To the homogenized mixture an amount of 38.61 grams of acrylate copolymer (Duro-Tak® 387-2287, solid content 50.5%) was added and mixed well. The wet adhesive formulation was coated on a release liner and dried to give a dry coat weight of 5 mg/cm².

### Preparation of Final Five Layer Laminate of Donepezil TDS, Die-Cut, and Pouching

A polypropylene rate controlling membrane (Celgard® 2400) pretreated with the membrane treatment composition was laminated onto the adhesive side of the drug reservoir. Then the contact adhesive was laminated on top of the rate controlling membrane laminated with drug reservoir. The release liner on the drug reservoir side was replaced with a backing film. The final five layer laminate was die-cut into patches and each test patch was pouched individually. The resultant transdermal delivery system comprised a drug reservoir layer and a contact adhesive layer with a rate controlling microporous membrane layer situated between the drug reservoir and the contact adhesive layers, as depicted in FIG. 1A.

The composition of the donepezil transdermal delivery system (TDS) of the present example is summarized in the Table 9.1.

TABLE 9.1
Donepezil HCl TDS With Pretreated Microporous Membrane

| Layer | Ingredient | Trade Name | % w/w | |
|---|---|---|---|---|
| Overlay | Woven Polyester | KOB 052 | 15 | mil |
| | Fabric | | | |
| | Acrylate adhesive | Duro-Tak 87- | 8 | mg/cm2 |
| | | 2052/2287/2051 | | |
| Separating | Polyester Laminate | Scotchpak 1012 | 2 | mil |
| Layer | | | | |
| Drug | Donepezil | N/A | 16.0% | |
| Reservoir | hydrochloride | | | |
| (Coat weight: | Sodium bicarbonate | N/A | 2.6% | |
| 12 mg/cm²) | Triethyl citrate | N/A | 10.0% | |
| | Glycerine | N/A | 10.0% | |
| | Lauryl lactate | Ceraphyl 31 | 3.0% | |
| | Sorbitan laurate | SPAN 20 | 2.0% | |
| | Crospovidone | Kollidon CL-M | 15.0% | |
| | Ascorbic palmitate | N/A | 0.5% | |
| | Acrylic adhesive- | Duro-Tak 87- | 40.9% | |
| | Duro-Tak 87-2287 | 2287 | | |
| | Total | | 100% | |
| Microporous | Microporous | Celgard 2400 | 1 | mil |
| Membrane | polypropylene | | | |
| (Vehicle coat | membrane | | | |
| weight: | Triethyl citrate | N/A | 66.7% | |
| 1.11 mg/cm²) | Lauryl lactate | Ceraphyl 31 | 20.0% | |
| | Sorbitan laurate | SPAN 20 | 13.3% | |
| | Total | | 100% | |
| Contact | Triethyl citrate | N/A | 10.0% | |
| Adhesive | Lauryl lactate | Ceraphyl 31 | 3.0% | |
| (Coat weight: | Sorbitan laurate | SPAN 20 | 2.0% | |
| 5 mg/cm²) | Crospovidone | Kollidon CL-M | 20.0% | |
| | Acrylate adhesive | Duro-Tak 87- | 65.0% | |
| | | 2287 | | |
| | Total | | 100% | |
| Release | Silicone coated | | 3 | mil |
| Liner | polyester film | | | |

Control samples of Donepezil TDS were prepared in the same way using un-treated Celgard® 2400 membrane instead of the treated membrane.

After equilibration for two weeks at room temperature, in vitro skin flux from the patches were tested as follows:

### Preparation of Skin

Dermatomed human cadaver skin was obtained from a skin bank and frozen until ready for use. The skin was placed in water at 60° C. for 1-2 minutes after thawing and the epidermis carefully separated from dermis. The epidermis was either used immediately or wrapped and frozen for later use.

### In Vitro Skin Flux Test

In vitro skin flux studies were performed using a Franz type diffusion cell with an active diffusion area of 0.64 cm². The epidermis was mounted between the donor and receptor compartments of the diffusion cell. The patch was placed over the skin and the two compartments were clamped tight together.

The receptor compartment was filled with 0.01M phosphate buffer, pH 6.5, containing 0.01% gentamicin. The solution in the receptor compartment was continually stirred using a magnetic stirring bar in the receptor compartment. The temperature was maintained at 32°±0.5° C. Samples were periodically drawn from receptor solution and drug content analyzed using high performance liquid chromatography (HPLC).

The results were calculated in terms of amount of drug diffused through the epidermis per cm² per hour.

The results are plotted in FIG. 7. Each data point is the average of three skin donors, four replicates per donor. The patch with the untreated membrane shows lower flux profile even after 2 weeks' equilibration.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1.-16. (canceled)

17. A transdermal delivery system, comprising:
a skin contact adhesive layer to attach the system to the skin of a user;
a drug reservoir layer comprising a salt form of an active agent, sodium bicarbonate, glycerine, ascorbic palmitate, and a drug carrier composition; and
a microporous membrane disposed between the adhesive layer and the drug reservoir layer, the microporous membrane comprising a plurality of pores filled with a membrane treatment composition,
wherein the salt form of an active agent is donepezil hydrochloride.

18. The system of claim 17, wherein the plurality of pores are filled with the membrane treatment composition prior to the microporous membrane being disposed between the adhesive layer and the drug reservoir layer.

19. The system of claim 18, wherein the drug carrier composition and the membrane treatment composition are different.

20. The system of claim 19, wherein the membrane treatment composition comprises a nonionic surfactant, a long-chain aliphatic alcohol, a citric acid ester, or combinations thereof.

21. The system of claim 20, wherein the nonionic surfactant is sorbitan monolaurate, the long-chain aliphatic alcohol is lauryl lactate or octyldodecanol, and the citric acid ester is triethyl citrate.

22. The system of claim 19, wherein the drug carrier composition comprises a nonionic surfactant, a long-chain aliphatic alcohol, a citric acid ester, or combinations thereof.

23. The system of claim 22, wherein the nonionic surfactant is sorbitan monolaurate, the long-chain aliphatic alcohol is lauryl lactate or octyldodecanol, and the citric acid ester is triethyl citrate.

24. The system of claim 17, wherein the skin contact adhesive layer comprises a contact adhesive layer drug carrier composition.

25. The system of claim 24, wherein the contact adhesive layer drug carrier composition comprises a nonionic surfactant, a long-chain aliphatic alcohol, a citric acid ester, or combinations thereof.

26. The system of claim 24, wherein the contact adhesive layer drug carrier composition is different from the drug carrier composition.

27. The system of claim 17, wherein the drug reservoir layer comprises about 5 wt % to about 25 wt % donepezil hydrochloride.

28. The system of claim 27, wherein the drug reservoir layer comprises about 16.0 wt % donepezil hydrochloride.

29. The system of claim 17, wherein the drug reservoir layer comprises between about 2-5 wt % sodium bicarbonate.

30. The system of claim 29, wherein the drug reservoir layer comprises between about 2.6 wt % sodium bicarbonate.

31. The system of claim 17, wherein the drug reservoir layer comprises about 0.1 wt % to about 2 wt % of ascorbic palmitate.

32. The system of claim 31, wherein the drug reservoir layer comprises about 0.5 wt % ascorbic palmitate.

33. The system of claim 32, wherein the drug reservoir layer comprises about 16.0 wt % donepezil hydrochloride;
about 2.6 wt % sodium bicarbonate;
about 10.0 wt % triethyl citrate;
about 3.0 wt % lauryl lactate;
about 2.0 wt % sorbitan lactate;
about 10.0 wt % glycerine;
about 15.0 wt % crosslinked polyvinylpyrrolidone;
about 0.5 wt % ascorbic palmitate; and
about 40.9 wt % copolymer of acrylic acid and vinyl acetate.

34. The system of claim 17, wherein the membrane treatment composition comprises between about 10-17 wt % sorbitan monolaurate, between about 15-25 wt % lauryl lactate, and between about 60-75 wt % triethyl citrate.

35. The system of claim 17, wherein the contact adhesive layer comprises sorbitan monolaurate, lauryl lactate, and triethyl citrate.

36. A method for treating Alzheimer's disease, comprising:
applying to skin of a subject a transdermal delivery system according to claim 1, whereby said applying generates a base form of the donepezil hydrochloride for delivery to the skin.

37. A method for transdermal delivery of a base form of donepezil, comprising:
providing a transdermal delivery system according to claim 17,
securing, or instructing to secure, the system to the skin of a user to deliver the base form of the donepezil from the system to the skin, whereby (i) the time to reach steady state flux is at least about 20% faster compared to a system with no membrane treatment composition in the pores of the microporous membrane, (ii) the system achieves its steady state equilibrium flux at least 20% faster compared to a system with no membrane treatment composition in the pores of the microporous membrane; and/or (iii) the donepezil diffuses from the system to the skin at least 20% faster compared to a system with no membrane treatment composition in the pores of the microporous membrane.
